(19) United States
(12) Patent Application Publication
(10) Pub. No.: US 2026/0056198 A1
(43) Pub. Date: Feb. 26, 2026

(54) AIR-SEGMENTED INJECTION OF FLUIDS IN A FLOW CELL FOR SPR ANALYSIS

(71) Applicant: CYTIVA SWEDEN AB, Uppsala (SE)

(72) Inventors: Henrik Berling, Uppsala (SE); Linnéa Nygren Babol, Uppsala (SE)

(21) Appl. No.: 19/105,897

(22) PCT Filed: Aug. 16, 2023
(86) PCT No.: PCT/EP2023/072618

§ 371 Date: Feb. 24, 2025

(30) Foreign Application Priority Data
Aug. 25, 2022 (GB) 2212380.6
Sep. 21, 2022 (GB) 2213770.7

(51) Int. Cl. G01N 33/573; G01N 33/543; G01N 33/569

(52) U.S. Cl. CPC G01N 33/573; G01N 33/54373; G01N 33/56983; G01N 2333/165; G01N 2333/9123; G01N 2333/948

(57) ABSTRACT

According to various aspects, the present invention provides a method of delivering a plurality of sample solutions to a sensor surface (102) for analysis. It can additionally, or alternatively, provide systems and methods for enabling improved investigation of analyte behaviours. The sensor (102) may, for example, be used to determine kinetic binding properties of analytes (such as proteins) to surface bound ligands. In one aspect, a method comprises: a) introducing (902) a first sample solution (502) into a first channel (506), the first channel (506) configured to deliver sample solution to the sensor surface (102); b) introducing (904) a second sample solution (504) into the first channel (506); c) flowing (906) the first sample solution and the second sample solution through the first channel (506) and over the sensor surface (102); and d) detecting (912) the presence or absence of binding of analyte from at least one of the sample solutions (502, 504) at the sensor surface (102), wherein no running buffer solution is passed over the sensor surface (102) throughout steps a)-d).

### TECHNICAL FIELD

The present disclosure relates to analysis of sample solutions at a sensor surface, and more particularly to systems and methods for delivering a plurality of sample solutions to said sensor surface and/or for enabling improved investigation of analyte behaviours.

### BACKGROUND

Analytical sensor systems that can monitor interactions between molecules, such as biomolecules, in real time are gaining increasing interest. Such systems typically enable one or more of binding, kinetics, affinity, specificity and concentration of molecules (‘analytes’) contained in sample solutions to be determined. Optical biosensors are particularly useful for this purpose and are usually referred to as interaction analysis sensors or biospecific interaction analysis sensors. A representative such biosensor system is the BIACORE® instrumentation sold by Cytiva, which uses surface plasmon resonance (SPR) for detecting interactions between molecules at a sensing surface without any need for labels. As a respective sample is passed over the sensor surface, the progress of binding can be measured and provides a direct reflection of the rate at which an interaction between molecules is occurring.

A typical output from systems such as the BIACORE® system is a graph or curve describing the progress of a molecular interaction with time, including an association phase part and a dissociation phase part. This binding curve, which is usually displayed on a computer screen, is often referred to as a ‘sensorgram’. With the BIACORE® system (and analogous sensor systems) it is thus possible to determine in real time without the use of labelling, and often without purification of the substances involved, not only the presence and concentration of a particular analyte in a sample, but also additional interaction parameters, including kinetic rate constants for binding (association) and dissociation in the molecular interaction as well as the affinity for the interaction being assessed. The association rate constant (ka) and the dissociation rate constant (kd) can be obtained by fitting the resulting kinetic data for a number of different sample analyte concentrations to mathematical descriptions of interaction models in the form of differential equations. The affinity (expressed as the affinity constant KA or the dissociation constant K_{D}) can be calculated from the association and dissociation rate constants. It is also possible to measure affinity values by equilibrium binding analysis, which involves determining, for a series of analyte concentrations, the level of binding at equilibrium, or steady state, which is presumed to have been reached at or near the end of the association phase of the binding interaction.

Traditionally, such analysis involves analysing interaction between a molecule bound at the sensor surface (often referred to as a ‘ligand’) and a molecule of interest (often referred to as an ‘analyte’) in an injected sample solution. In this traditional approach, running buffer is passed over the sensor surface between injections of different sample solutions. This running buffer is typically composed to mimic physiological conditions to stabilize ligands and analytes. A typical buffer solution comprises a component such as Hepes, TRIS or phosphate ions to keep pH close to 7.4, sodium chloride at 150 mM, 3 mM EDTA and 0.05% of the detergent P20.

The present inventors have identified, however, that in some cases it would be useful to analyse not just the interaction of an analyte with an immobilised ligand, but also or alternatively the interaction between two or more successively injected analytes or sample compositions. For example, it would be useful to inject a first sample solution containing a first analyte, followed by a second sample solution containing a second analyte, and determine how and whether the first and second analytes interact. However, existing systems and methods do not effectively facilitate such ‘polyinjection’ analysis. In particular, the present inventors have identified that the use of running buffer between samples in existing systems makes such analysis difficult, because the first analyte is washed away, dissociates or is disturbed before the second analyte can reach the sensor surface.

The present inventors have also identified that current analysis methodologies do not always enable accurate and effective analysis of sample behaviours. In particular, current methodologies are poorly optimised for detecting binding signals from analytes which bind weakly when forming a complex with another analyte (or ligand) that produces a significantly stronger binding response. Current methodologies are also poorly optimised for determining whether a binding response signal is representative of a given reaction or binding event of interest. In more general terms, existing sample assay methodologies are limited in their applications and effectiveness.

It would be advantageous to provide systems and methods which address one or more of the above-described problems, in isolation or in combination. In particular, it would be advantageous to provide improved sample assay methodologies and systems.

### SUMMARY

This overview introduces concepts that are described in more detail in the detailed description. It should not be used to identify essential features of the claimed subject matter, nor to limit the scope of the claimed subject matter.

According to one aspect of the present disclosure, there is provided a method of delivering a plurality of sample solutions to a sensor surface for analysis, comprising: a) introducing a first sample solution into a first channel, the first channel configured to deliver sample solution to the sensor surface; b) introducing a second sample solution into the first channel; c) flowing the first sample solution and the second sample solution through the first channel and over the sensor surface; and d) detecting the presence or absence of binding of analyte from at least one of the sample solutions at the sensor surface. No running buffer solution is passed over the sensor surface throughout steps a)-d).

This method provides a robust and reliable mechanism for performing ‘polyinjection’ analysis, in other words analysis involving two or more sequentially introduced sample solutions in the absence of running buffer. “Running buffer” in this context comprises a continuous flow of buffer, provided from a running buffer reservoir, that can be introduced into the system and flows over the sensor surface to wash away analyte. Such running buffer is thus to be contrasted with the sample solutions introduced into the system. Unlike continuous running buffer, sample solutions in this context are discrete, highly controlled volumes of fluid which are provided to enable the behaviour of analytes at the sensor surface to be studied. Sample solutions are typically provided from sample vials or microplate wells. Preventing running buffer from entering the system during analysis (e.g. until all sample solutions have been sequentially injected and analysed) avoids any interference with such sample solutions or the sensor surface by the running buffer. It will be appreciated that the disclosed method is not limited to only two sample solutions. Rather, any number of sequential samples can be provided.

Preferably, the second sample solution is separated from the first sample solution in the first channel by a gas segment. More generally, if more than two sample solutions are used, each can be separated from any preceding sample in the first channel by a respective gas segment. Use of a gas segment between successive sample solutions ensures that there is no (or minimal) mixing between the subsequent solutions in the first channel. This means that the samples only interact once they reach (or are near) the sensor, meaning that this interaction takes place at the sensor and can be analysed. Note that the term ‘interaction’ is used synonymously with the term ‘reaction’ herein.

Preferably, the gas segment is extracted from the first channel via a second channel prior to the gas segment reaching the sensor surface, such that the gas segment does not contact the sensor surface.

This is advantageous because in some cases if the gas segment is allowed to reach the sensor surface, it can interfere with the measurement and produce unwanted signal or noise. The gas segment can also disturb or even damage the sensor surface (or a ligand or analyte immobilised/captured there). Extracting the gas segment prior to it reaching the sensor surface prevents this, whilst still enabling the gas segment to prevent mixing of the samples throughout most of the samples' journey through the first channel towards the sensor. By virtue of the disclosed method, a reliable mechanism for conducting polyinjection analysis is thus provided. In particular, behaviour of the first and second sample solutions (typically of analytes contained therein) at the sensor surface can be reliably analysed.

Preferably, introducing the first and second sample solutions into the first channel comprises aspirating the first and second sample solutions from respective first and second sample solution reservoirs and injecting the sample solutions into the first channel. Such a mechanism enables accurate and reliable introduction of a specific volume of sample solution into the system. Typically, the sample solution reservoirs comprise vials having stoppers which are pierced by a needle during aspiration and injection. Other sample solution reservoirs can be used, however.

Preferably, steps a)-d) described above are performed using a first pump configured to introduce sample solutions into the first channel and cause samples to flow through the first channel and over the sensor surface. This first pump may be contrasted to a distinct running buffer pump, which may comprise a separate pump configured to introduce continuous running buffer into the system. Use of different pumps to introduce sample solutions and running buffer enables accurate polyinjection analyses to be conducted. In particular, the sample analysis pump can be operated during analysis, while the running buffer pump is kept inactive. This enables the introduction of continuous running buffer during analysis to be prevented, which has the advantages described above. Once analysis is complete, the sample analysis pump can be deactivated and the running buffer pump can be activated, to clean out the system and sensor surface and reset the reading to a baseline value. In particular, prior to step a) and/or after step d) described above, running buffer obtained from a running buffer reservoir is preferably caused to pass over the sensor surface by the running buffer pump.

Preferably, the gas segment is extracted at most 40 seconds prior to the second sample solution reaching the sensor surface. This time threshold ensures that there is a sufficiently short time window during which the sample solutions can mix before they reach the sensor surface. This is beneficial, because a short time window ensures that mixing (and thus interaction) between the sample solutions prior to reaching the sensor surface is negligible. This ensures that the reaction occurs at the sensor surface and can thus be analysed. A short mixing window before the sensor surface is reached is particularly important for the study of fast reactions, such as various examples described in the Implementations section below.

As noted above, the disclosed method can be performed for more than two sample solutions. Preferably, the method is performed for three or more sample solutions, with each sample solution separated from any preceding sample solution in the first channel by a respective gas segment. This allows interaction between a greater number of sample solutions to be analysed, which in turn enables more sophisticated applications of the disclosed technology, as described in the Implementations and Examples below.

The sensor surface may have a ligand immobilized thereto and step d) described above may comprise detecting the presence or absence of binding of analyte from at least one sample solution to the ligand on the sensor surface. This enables analysis of how subsequent samples interact with the ligand, particularly in the presence of another analyte, to be performed more effectively.

Step d) may additionally or alternatively comprise identifying whether an analyte comprised in one of the sample solutions binds to an analyte comprised in another of the sample solutions that has previously bound to the sensor surface. For example, a first analyte from the first sample may bind to the sensor surface, and step d) may then comprise identifying whether a second analyte from the second sample solution binds to the analyte from the first sample solution. Such analysis enables study of how subsequent samples interact with one another to be performed more effectively.

Preferably, the first and second sample solutions are introduced into the first channel in a first order, and the method further comprises repeating the method steps with the first and second sample solutions introduced into the first channel in a second order different to the first order. For example, sample A may be introduced, followed by sample B, in the manner described herein. The interaction between A and B at the sensor surface may then be analysed. The method may then be repeated, but this time with sample B introduced first, followed by sample A. This enables analysis of how the samples may interact when arriving at a ligand or sensor surface in a different order. This can inform analysis of preferred binding sites of analytes.

Whilst some implementations of the disclosed methodology involve injecting a plurality of samples each containing a respective analyte, in some cases it can be beneficial to inject one or more samples not containing an analyte. For example, the first sample solution may have a first pH value and comprise a first analyte and the second sample solution may have a second pH value and not comprise an analyte. In an example of this implementation, the first sample may comprise a particular form of buffer solution having a particular pH value, e.g. 7.4. The first sample solution contains a first analyte of interest configured to bind to a ligand (or other analyte) at the sensor surface. The second sample may then comprise the same form of buffer solution with a different pH value, e.g. 6.0 but not containing an analyte. When the second sample solution flows over the sensor surface, it can then be determined how the change of pH affects the analyte that has bound at the sensor surface. This enables more sophisticated analysis of how analyte behaviour is impacted by the pH environment to be conducted.

The first and second sample solutions may have first and second respective concentrations, and the method may further comprise modifying the concentration of at least one of the first and second sample solutions and repeating the method steps with the modified solution(s). This enables analysis of how analyte interaction is impacted by the analyte concentration to be conducted.

Each sample solution may comprises a different respective analyte or may comprises the same analyte.

As noted above, at least one sample solution may comprise buffer. A sample solution of buffer is to be contrasted with continuous flow of running buffer. In particular, a sample solution of buffer comprises a specified volume of buffer provided via a sample injection pump or similar sample injection mechanism, typically from a sample vial, in the same way as any other sample solution. A buffer sample is injected in the context of analysing analyte behaviour at the sensor surface. This is in contrast to a flow of running buffer which is provided from a buffer reservoir, by a buffer reservoir pump, and which has the sole purpose of ‘flushing out’ and wetting the system before or after an assay has been performed.

In contrast to an unspecified volume of running buffer, an accurately measured buffer sample (also referred to as a ‘buffer pulse’ or ‘blank sample’) provided as a sample solution in the context of the present method can be used to conduct various forms of analysis more effectively, as discussed in the Implementations and Examples below. For example, use of buffer samples can inform how the absence of a particular analyte impacts binding responses, can be used to detect response signals which would otherwise be swamped by other signals, and can be used to assess kinetics of analytes. In one illustrative example, an assay may be performed with two successive sample solutions comprising first and second analytes respectively. The assay may then be followed up with a second assay where the first sample is a blank (contains no analyte) and the second sample contains the same analyte as the second sample in the first assay. The binding observed in the first and second assays may then be compared. If binding of the second sample occurs in both assays then it has been determined that the second analyte binds to the ligand. If binding of the second sample only occurs in the first assay but not in the second assay, on the other hand, then it has been determined that the second analyte only binds in the presence of the first analyte, i.e. does not bind directly to the ligand. In that case it may be said that binding of the second analyte is dependent on the first analyte. This and many other example analyses become possible through use of a blanks or buffer sample within an assay.

At least one sample solution may comprise at least one of: an antibody; an antigen; an enzyme; an agonist; an antagonist; a wash solution; or a sensor surface regeneration solution.

The first sample solution may contact the sensor surface during a first time period, and the second sample solution may contact the sensor surface during a second time period. The first and second time periods may be referred to as the ‘contact times’ of the first and second samples. The delay between the end of the first time period and the start of the second time period (i.e. the gap between the two contact times) is preferably 40 seconds or less. This time threshold ensures that the disclosed methodology is suitable for analysing interactions involving analytes that dissociate or degrade quickly. For example, a first injected analyte in a first sample may bind to the sensor surface, but then dissociate soon after. Ensuring that the second sample arrives within 40 seconds or less ensures that for a wide range of implementations the second sample will arrive in time for the interaction of interest to occur at the sensor, before the first analyte dissociates. More preferably, the delay between the first and second sample is 30 seconds or less, 20 seconds or less, 10 seconds or less, or 1 second or less. These successively shorter time periods ensure that even very fast dissociating analytes can be analysed. The range of times is made possible through the methodologies and systems disclosed herein because there is no ‘down-time’ due to intermediate running buffer injection. The precise time between first and second samples arriving at the sensor can be determined by the length of flow channel that the samples flow through between gas segment extraction and the sensor, for example. The disclosed methodologies and available range of times between samples means the assay environment can be developed around and modified to suit particular analytes of interest.

More generally, the methods disclosed herein are particularly useful for analysing analytes which interact quickly. This is because mixing in the first channel is prevented until the samples are near the sensor surface. The short-lived interactions thus take place at and can be detected and analysed at the sensor surface. Also, where binding occurs to a ligand at the sensor surface, the lack of an intermediate running buffer step between sample injections means that the next sample can be provided before the analyte of the first sample has dissociated, partly or entirely, from the sensor surface, which is important for analysing various types of interaction.

The first sample solution may comprise a membrane protein and the second sample solution may comprise a lipid configured to at least partially cover the membrane protein. Such membrane proteins may be particularly susceptible to disturbance by running buffer flow. Hence, analysis of such membrane proteins and their interaction with lipids is particularly well served by the methods of the present disclosure which can avoid running buffer being present throughout the analysis.

The volume of at least one sample solution introduced into the first channel may be determined based on a target duration during which the at least one sample solution is present at the sensor surface. For example, based on a known or pre-determined flow rate of the sample and a pre-determined target duration during which time the sample is to be present at the sensor surface, the required volume of sample can be determined.

The method may further comprise identifying a complex that is built up on the sensor surface, said complex comprising analytes from at least two of the sample solutions. Analysis of complexes which require analytes to be delivered quickly (e.g. because of fast dissociation times of the analytes involved) are particularly well served by the methods of the present disclosure which can avoid running buffer delaying the provision of the next sample to the sensor surface.

Similarly, the method may further comprise identifying whether respective analytes in the first and second sample solutions compete for binding sites (either at a ligand or at an analyte previously bound to the sensor surface). In that case, it is important that the second analyte arrives while the first analyte is still substantially present at the surface (otherwise it will not be possible to accurately determine whether there is competition). This is facilitated by the presently disclosed methods which enable the second sample to arrive soon after the first because there is no wait during which running buffer is injected. Also, the precise timing of the samples arriving at the sensor surface can be calibrated through the length of the first flow channel, for example.

The method may further comprise identifying whether analyte comprised in at least one of the sample solution binds to a same or different epitope as analyte comprised in another of the sample solutions. Such binding characteristics can be analysed with particular ease when using the disclosed methods, because interference by running buffer flow is avoided.

Step d) of the above-described method may be based on evanescent wave sensing or surface plasmon resonance (SPR).

The gas segment may comprise air, nitrogen or another inert gas that does not affect the sample solutions. Use of air is typically the most straightforward and causes the fewest complications.

The sensor surface may comprise silver or gold. While the use of a silver sensor surface may provide optimum sensor performance, silver has a tendency to degrade over time. Gold is more resilient and does not deteriorate over time in water solutions. It is also simple to attach organic molecules to gold.

The first and second channels may form a substantially T-shaped junction which provides good performance. Any other junction shape may be used, as long as there are at least three directions for flow: from the sample reservoir; to the sensor; and an exit channel for the gas segment.

According to another aspect of the present disclosure, there is provided a sample analysis system comprising a first channel and a sensor surface. The sample analysis system is configured to perform the steps of: a) introducing a first sample solution into the first channel, the first channel configured to deliver sample solution to the sensor surface; b) introducing a second sample solution into the first channel; c) flowing the first sample solution and the second sample solution through the first channel and over the sensor surface; and d) detecting the presence or absence of binding of analyte from at least one of the sample solutions at the sensor surface, wherein no running buffer solution is passed over the sensor surface throughout steps a)-d).

More generally, a sample analysis system configured to perform any of the methods disclosed herein is disclosed. Note that the terms ‘biosensor system’ and ‘sample analysis system’ are used interchangeably herein. The terms “sample” and “sample solution” are also used interchangeably herein and refer to a specified volume of fluid, typically containing an analyte of interest, suitable for introducing into the system during sample analysis at the sensor surface.

According to yet another aspect of the present disclosure, there is provided a method of delivering a plurality of sample solutions to a sensor surface for analysis, comprising: a) introducing a first sample solution into a first channel, the first channel configured to deliver sample solution to the sensor surface; b) introducing a second sample solution into the first channel, wherein the second sample solution is separated from the first sample solution in the first channel by a gas segment; c) flowing the first sample solution, second sample solution and gas segment through the first channel towards the sensor surface; d) prior to the gas segment reaching the sensor surface, extracting the gas segment from the first channel via a second channel such that the gas segment does not contact the sensor surface; e) flowing the first sample solution and the second sample solution over the sensor surface; and f) detecting the presence or absence of binding of analyte from at least one of the sample solutions at the sensor surface. Such a method enables accurate analysis of multiple sample solutions in succession. Mixing of the sample solutions is prevented as described above. Preferably, no running buffer is provided into the system through steps a)-f) which has the associated benefits described above.

According to one further aspect of the present disclosure, there is provided a method of assaying multiple sets of sample solution, comprising providing a first set of sample solutions, wherein the first set of sample solutions comprises at least three sample solutions. The method further comprises obtaining a first binding signal by performing an assay with the first set of sample solutions during which each sample solution in the first set of sample solutions is flowed sequentially over a sensor surface.

The term “sequentially” in this context means one after another, optionally with intermediate gas segments and/or with running buffer or other wash solutions provided between samples, as described more fully below.

It will be appreciated that when each sample solution in the first set of sample solutions flows over the sensor surface, it will generate a respective response signal indicative of the presence (or absence) of binding of analyte in that sample solution at the sensor surface. For example, a solution comprising an analyte that binds strongly to a ligand or other analyte present at the sensor surface will generate a large response signal. A solution comprising an analyte that binds weakly will generate a small response signal, while a sample containing no analyte (e.g. a buffer sample) or containing an analyte that does not bind will generate no binding response signal. The “first binding signal” of the first set of sample solutions as defined herein thus comprises a combination of these successive individual response signals produced by the respective sample solutions in the first set of sample solutions during the assay. The first binding signal may thus be considered a total or composite binding signal of the first set of sample solutions.

The method further comprises providing a second set of sample solutions, wherein the second set of sample solutions comprises a subset of the first set of sample solutions and a buffer sample solution. In the present disclosure the term “subset” means “the same solutions but at least one fewer”. Thus, if the first set of sample solutions comprises solutions A, B and C, then the second set of sample solutions may comprise solutions A and B but not C, or A and C but not B etc. In place of the omitted sample solution, a sample of buffer solution is provided in the second set of sample solutions.

A “buffer sample” (also referred to as a ‘buffer pulse’ or ‘blank sample’) as defined herein is a sample solution, provided in the same way as any other sample solution from a sample reservoir during an assay, but containing only buffer and no analyte. The buffer sample therefore does not result in a binding response signal when it reaches the sensor surface. As described below, use of a buffer sample during an assay can yield important insights into binding characteristics of other analytes in the assay. Note that a buffer sample is to be contrasted with running buffer. As defined herein, running buffer is provided from a running buffer reservoir via a different mechanism to sample solutions (e.g. via a discrete running buffer pump as shown in FIG. 4 below) and has the sole purpose of washing out the analytes at the end of an assay and keeping the instrumentation wetted when no sample is flowing. This is in contrast to a buffer sample which is provided like any other sample from a sample reservoir and which has a discrete, highly controlled volume and is injected for the purpose of analysing the behaviour of analytes at the sensor surface.

The method further comprises obtaining a second binding signal by performing an assay with the second set of sample solutions during which each sample solution in the second set of sample solutions is flowed sequentially over the sensor surface. As before, the “second binding signal” is here made up of a combination of the successive individual response signals produced by the respective sample solutions in the second set of sample solutions during the assay. The second binding signal may thus be considered a total or composite binding signal of the second set of sample solutions.

The method further comprises generating a third binding signal by subtracting the second binding signal from the first binding signal. The third binding signal may thus be considered as a difference signal indicating the difference between the first and second binding signals. The third binding signal may be displayed for analysis by a user or used by the system for computational analysis. As will be described more fully below, the third (or difference) binding signal can reveal previously hidden analyte behaviours and reveal interactions and relationships that are not apparent from the first or second binding signals alone. The disclosed methodology thus provides improved methodologies for sample assay which allows for more effective and varied investigations to be conducted into analyte behaviour.

Providing the first and/or second set of sample solutions may comprise introducing the respective sample solutions into a channel configured to deliver sample solution to the sensor surface. Preferably, each sample solution introduced into the channel is separated from any preceding sample solution in the channel by a respective gas segment. This prevents mixing between the samples before they reach the sensor surface, allowing more effective analysis as discussed more fully below. Preferably, prior to each gas segment reaching the sensor surface, the method comprises extracting the respective gas segment from the channel via another channel such that each respective gas segment does not contact the sensor surface. This ensures that the gas segment does not damage the sensor surface, as described more fully below, whilst still enabling the gas segment to prevent mixing of the samples throughout most of the samples' journey through the first channel towards the sensor. By virtue of the disclosed method, a reliable mechanism for conducting sample assays is thus provided. In particular, behaviour of sample solutions (or, more precisely, the analytes contained therein) at the sensor surface can be reliably analysed.

Preferably, introducing the first and/or second set of sample solutions comprises aspirating the respective sample solutions from respective sample solution reservoirs and injecting the sample solutions into the channel. Such a mechanism enables accurate and reliable introduction of a specific volume of sample solution into the system. Typically, the sample solution reservoirs comprise vials having stoppers which are pierced by a needle during aspiration and injection. Other sample solution reservoirs can be used, however.

Obtaining the first and/or second binding signal may comprise detecting the presence or absence of binding involving analytes from the respective sample solutions at the sensor surface. For example, the sensor surface may have a ligand immobilized thereto. In that case, obtaining the first and/or second binding signal may comprise detecting the presence or absence of binding of analyte from at least one sample solution to the ligand on the sensor surface. This enables analysis of how subsequent samples interact with the ligand, particularly in the presence of another analyte, to be performed more effectively.

Alternatively or additionally, obtaining the first and/or second binding signal may comprise detecting the presence or absence of binding between analytes from two or more of the sample solutions at the sensor surface. This enables analysis of how subsequent samples interact with one another to be performed more effectively.

In one advantageous implementation, the first set of sample solutions comprises a first sample solution and a second sample solution, wherein, when the second sample solution reaches the sensor surface, the first sample solution generates a dissociation signal equal or greater than an association signal generated by the second sample solution, and wherein the second set of sample solutions does not comprise the second sample solution. In cases such as these, when the response decrease (dissociation) from the first sample solution is significant compared to the response increase (association) from the second sample solution, the response signal from the second sample solution will be obscured. In particular, the composite signal formed by the combination of the first sample dissociation and the second sample association will be flat (if the signals are equal) or be negative (if the first sample dissociation is greater than the second sample association). In such a scenario, it is virtually impossible to accurately analyse the (association) binding response of the second sample solution when using traditional analysis, because it is masked to such an extent by the (dissociation) binding response of the first sample solution. As a result, it can be impossible to accurately determine the presence or magnitude of binding of the second sample. The present methodology overcomes this problem, because the binding response signal of the first sample solution is effectively removed through subtraction of the second binding signal. The computed third (i.e. difference) binding signal thereby reveals the response of the second sample solution which would otherwise have been obscured. More effective sample analysis is therefore made possible.

In another advantageous implementation, the first set of sample solutions comprises a first sample solution and a second sample solution, wherein analyte in the second sample solution is capable of binding to analyte in the first sample solution and to a ligand on the sensor surface, and wherein the second set of sample solutions does not comprise the first sample solution. In traditional analysis, it would be difficult to determine whether a binding response signal associated with the second sample solution is the result of the second sample solution (or, specifically the analyte therein) binding to analyte in the first sample solution or to the ligand. This is typically addressed through introduction of a so-called “blocking” molecule which blocks binding to the ligand so that only binding between the sample solutions occurs and is detected. However, use of blocking is complex and time-consuming as described more fully below. The present methodologies enable the binding between the first and second sample solutions to be revealed without the use of blocking molecules, because binding between the second sample solution and ligand is effectively removed through subtraction. The third (i.e. difference) binding signal thereby contains only the signal relating to the interaction of interest between the first and second sample solutions. More effective sample analysis is therefore again made possible.

It will be appreciated that the terms “first” and “second” here are simply labels used to denote sample solutions in the first set of sample solutions. These terms therefore do not imply any absolute (or even relative) ordering of injection. For example, the “first” sample solution may in some cases be the second of three (or more) sample solutions injected from the first set of sample solutions.

In a further advantageous implementation, the first set of sample solutions comprises a first antibody, an antigen and a second antibody. In that case, the second set of sample solutions may not comprise the antigen. This enables binding between the second antibody and the ligand to be detected (in the second binding signal) and removed through subtraction. The third (i.e. difference) binding signal thereby reveals binding between the second antibody and the antigen, with “noise” from any binding of the second antibody to the ligand removed.

In one advantageous implementation, the method further comprises determining, based on the third binding signal, whether binding of analyte in a second sample solution in the first set of sample solutions is dependent on the presence of analyte from a first sample solution in the first set of sample solutions. This can be revealed, for example, by whether or not there is still a response signal left after subtraction of the second binding signal. This methodology allows more effective investigation of dependencies in binding, for example an investigation of whether a second sample requires the presence of a first sample to bind. It will again be appreciated that the terms “first” and “second” here are simply labels used to denote sample solutions in the first set of sample solutions and do not imply any absolute or relative ordering of injection. For example, in the assay one or more sample solutions may be injected before the “first” sample solution.

In another advantageous implementation, the method further comprises determining, based on the third binding signal, whether analyte in a first sample solution in the first set of sample solutions binds to a same epitope as analyte in a second sample solution in the first set of sample solutions. This can again be revealed, for example, by whether or not there is still a response signal left after subtraction of the second binding signal. This methodology allows more effective investigation of competition for epitopes between analytes, for example an investigation of whether a first sample binds to the same epitope as a second sample. This can be useful for identifying epitope competition between first and second antibodies, as well as between an antibody and another molecule such as a receptor. As above, it will once more be appreciated that the terms “first” and “second” here are simply labels used to denote sample solutions in the first set of sample solutions and do not imply any absolute or relative ordering of injection. For example, in the assay one or more sample solutions may be injected before the “first” sample solution.

Each sample solution may comprise a different respective analyte or may comprise the same analyte.

At least one sample solution may comprise at least one of: an antibody; an antigen; an enzyme; an agonist; an antagonist; a wash solution; or a sensor surface regeneration solution.

The volume of at least one sample solution introduced into the first channel may be determined based on a target duration during which the at least one sample solution is present at the sensor surface. For example, based on a known or pre-determined flow rate of the sample and a pre-determined target duration during which time the sample is to be present at the sensor surface, the required volume of sample can be determined.

Performing an assay with the first and/or second set of sample solutions may be based on evanescent wave sensing or surface plasmon resonance (SPR).

It will be appreciated that the methods disclosed herein are not limited to two assays. Rather, any suitable number of assays can be performed to generate a respective number of binding signals. These can then be subtracted from one another to generate the required number of difference signals. Examples involving more than two assays are described in more detail below.

According to another aspect of the present disclosure, there is provided a sample analysis system, comprising: a sensor having a surface over which sample solution can flow; and at least one channel configured to deliver sample solution to the sensor surface. The sample analysis system is configured to: provide a first set of sample solutions to the at least one channel, wherein the first set of sample solutions comprises at least three sample solutions; perform an assay wherein each sample solution in the first set of sample solutions is flowed sequentially over the sensor surface, in order to obtain a first binding signal; provide a second set of sample solutions to the at least one channel, wherein the second set of sample solutions comprises a subset of the first set of sample solutions and a buffer sample solution; perform an assay wherein each sample solution in the second set of sample solutions is flowed sequentially over the sensor surface, in order to obtain a second binding signal; and generate a third binding signal by subtracting the second binding signal from the first binding signal.

More generally, a sample analysis system configured to perform any of the methods disclosed herein is disclosed. Note that the terms ‘biosensor system’ and ‘sample analysis system’ are used interchangeably herein. The terms “sample” and “sample solution” are also used interchangeably herein and refer to a specified volume of fluid, typically containing an analyte of interest, suitable for introducing into the system during sample analysis at the sensor surface. References to binding of a sample or binding of a sample solution should be interpreted as references to binding of an analyte contained in the sample (solution).

According to yet another aspect of the present disclosure, there is provided a computer configured to cause a sample analysis system to perform any of the methods disclosed herein. In other words, any of the disclosed methods may be computer-implemented.

According to another aspect of the present disclosure, there is provided a computer-readable storage medium comprising instructions which, when executed by a computer, cause the computer to cause a sample analysis system to perform any of the methods disclosed herein.

### BRIEF DESCRIPTION OF THE FIGURES

Illustrative implementations of the present disclosure will now be described, by way of example only, with reference to the drawings. In the drawings:

FIG. 1 shows a schematic illustration of a biosensor system based on SPR;

FIG. 2 shows a representative sensorgram showing detector response versus time for an interaction between molecules taking place at the sensor surface;

FIG. 3 shows a schematic illustration of a Biacore T200 from Cytiva, which is an example of a biosensor system that can be used to implement the disclosed methods;

FIG. 4 shows a schematic illustration of a sample delivery system such as may be found within a biosensor system used to implement the disclosed methods;

FIG. 5 shows a sequence of four sample injections (A-D) analysed under existing methodologies whereby injection of continuous running buffer occurs between the injection of each sample;

FIG. 6 shows a sequence of four sample injections (A-D) analysed using the new and improved methodologies of the present disclosure, as a result of which the samples can be injected sequentially without intermediate running buffer in a process referred to herein as ‘polyinjection’;

FIG. 7a shows first and second sample solutions flowing through a channel, with mixing occurring at the sample interface;

FIG. 7b shows first and second sample solutions flowing through a channel, with mixing prevented by a gas segment;

FIGS. 8a-8c show an example mechanism for preventing a gas segment in the flow channel from contacting the sensor surface;

FIGS. 9a-9c show binding signals obtained by running an assay with three analytes injected in three respective orders;

FIGS. 10a and 10b shows another binding signal obtained by running an assay with three analytes, while FIG. 10b shows a closeup of part of the graph of FIG. 10a;

FIGS. 11a-11c show first and second binding signals obtained through respective assays, while FIG. 11c shows a difference curve obtained by subtracting the second binding signal from the first;

FIGS. 12a-12c show third and fourth binding signals obtained through respective assays, while FIG. 12c shows another difference curve obtained by subtracting the fourth binding signal from the third;

FIGS. 13a-13c show fifth and sixth binding signals obtained through respective assays, while FIG. 13c shows another difference curve obtained by subtracting the sixth binding signal from the fifth;

FIGS. 14a-14d shows seventh, eighth and ninth binding signals obtained through respective assays, while FIG. 14b shows a closeup of part of the graph of FIG. 14a. FIG. 14c shows a difference curve obtained by subtracting the eighth binding signal from the seventh binding signal, while FIG. 14d shows a difference curve obtained by subtracting the ninth binding signal from the seventh binding signal;

FIG. 15a shows schematically a complex that may be built up on a sensor surface comprising a ligand, two antibodies and an antigen;

FIGS. 15b and 15c show tenth and eleventh binding signals obtained through respective assays associated with the complex of FIG. 15a, while FIG. 15d shows another difference curve obtained by subtracting the eleventh binding signal from the tenth binding signal;

FIG. 16a shows a variety of difference signals obtained through the cycle subtraction methodology of the present disclosure;

FIG. 16b shows a summary table of epitope competition between three antibodies, the behaviour of which has been determined using the methodologies of the present disclosure;

FIG. 16c shows the results of the same procedure as FIG. 16a except performed at a sensor surface with a lower ligand density;

FIG. 17 shows the steps of a method for assaying multiple sets of sample solution;

FIG. 18 shows the steps of a method for delivering a plurality of sample solutions to a sensor surface for analysis; and

FIG. 19 shows the components of an example computer apparatus that can be used to implement the methods described herein.

Throughout the description and the drawings, like reference numerals refer to like features. The figures are not necessarily to scale.

### DETAILED DESCRIPTION

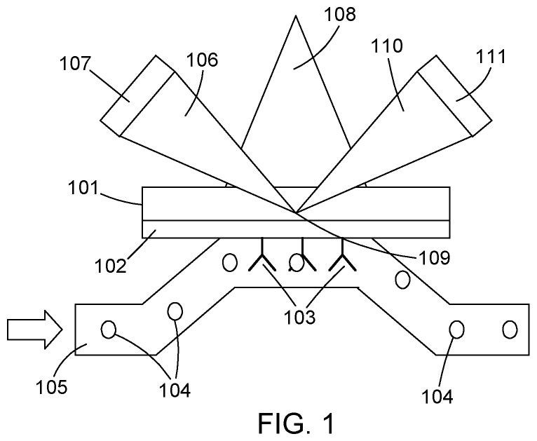

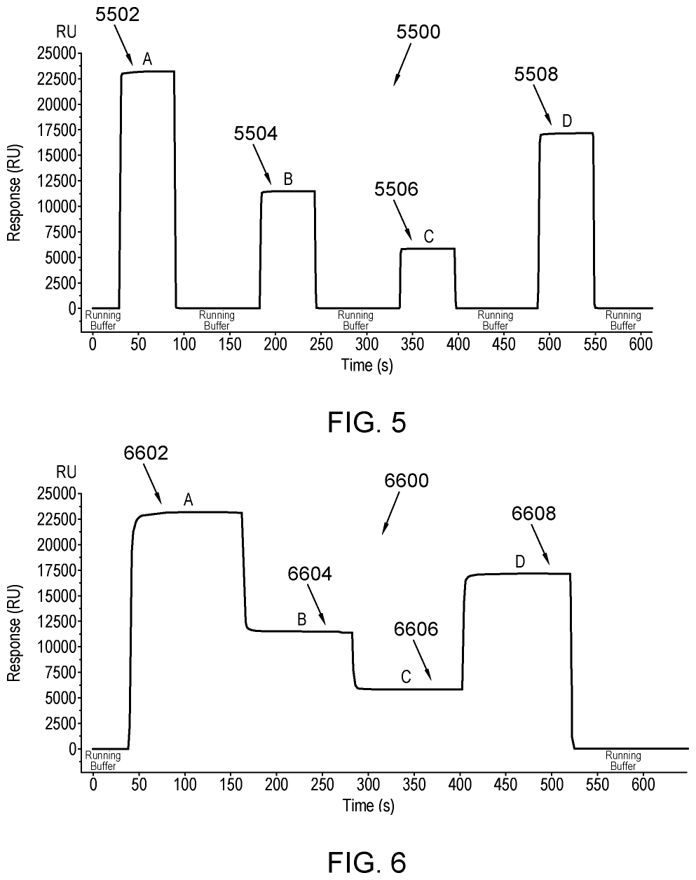

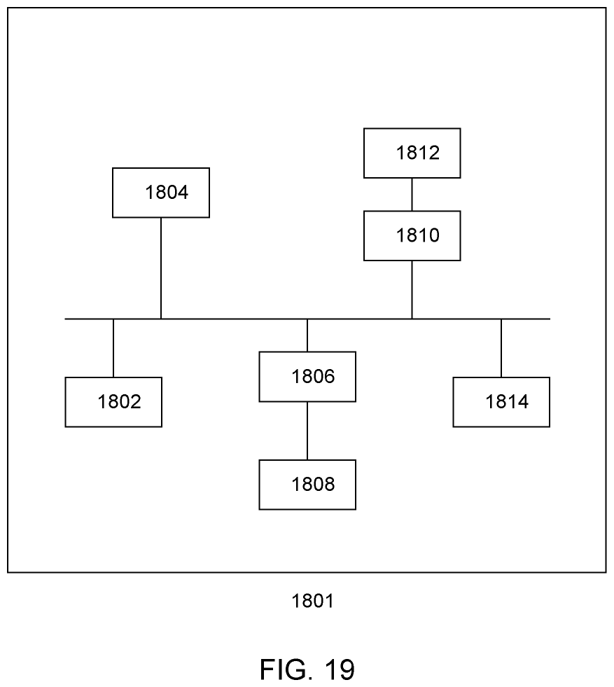

This detailed description describes, with reference to FIGS. 1-4, the fundamental principles of biosensors (also referred to as sample analysis systems) and their methods of operation. A traditional mechanism for analysing sample solutions at a biosensor is described with reference to FIG. 5. Improved methods for performing such analysis are then also described. Finally, with reference to FIG. 19, the components of an example computer apparatus that can be used to implement the methods described herein are described.

The systems and methods disclosed herein relate generally to delivering a plurality of sample solutions to a sensor surface for analysis. By preventing flow of running buffer between sample injection, samples can be delivered to the sensor surface sequentially without interference or delay. This mechanism of sequentially providing samples to the sensor surface without intermediate running buffer is referred to herein as a ‘polyinjection’ procedure or assay. Various features and methodologies for optimising such polyinjection assays are disclosed herein also. In particular, mixing of the sample solutions in the flow channel leading to the sensor surface can be substantially avoided due to the introduction of a gas segment to keep the sample solutions separate. Damage or disturbance at the sensor surface can be avoided by removal of the gas segment prior to the segment reaching the sensor surface. The disclosed systems and methods enable more effective interaction analysis and provide particular utility for a wide array of analyte binding analyses, as described more fully below.

Chemical sensors or biosensors are typically based on label-free techniques, detecting a change in a property of a sensor surface, such as e.g. mass, refractive index, or thickness for the immobilised layer. There are also other forms of sensor relying on some kind of labelling. Typical sensor detection techniques include, but are not limited to, mass detection methods, such as optical, thermo-optical and piezoelectric or acoustic wave methods (including e.g. surface acoustic wave (SAW) and quartz crystal microbalance (QCM) methods), and electrochemical methods, such as potentiometric, conductometric, amperometric and capacitance/impedance methods. With regard to optical detection methods, representative methods include those that detect mass surface concentration, such as reflection-optical methods, including both external and internal reflection methods, which are angle, wavelength, polarization, or phase resolved, for example evanescent wave ellipsometry and evanescent wave spectroscopy (EWS, or Internal Reflection Spectroscopy), both of which may include evanescent field enhancement via surface plasmon resonance (SPR), Brewster angle refractometry, bio-layer interferometry (BLI), critical angle refractometry, frustrated total reflection (FTR), scattered total internal reflection (STIR) (which may include scatter enhancing labels), optical wave guide sensors; external reflection imaging, evanescent wave-based imaging such as critical angle resolved imaging, Brewster angle resolved imaging, SPR-angle resolved imaging, and the like. Further, photometric and imaging/microscopy methods, “per se” or combined with reflection methods, based on for example surface enhanced Raman spectroscopy (SERS), surface enhanced resonance Raman spectroscopy (SERRS), evanescent wave fluorescence (TIRF) and phosphorescence may be mentioned, as well as waveguide interferometers, waveguide leaky mode spectroscopy, reflective interference spectroscopy (RIfS), transmission interferometry, holographic spectroscopy, and atomic force microscopy (AFR).

Commercially available biosensors include the afore-mentioned BIACORE® system instruments, manufactured and marketed by Cytiva, Uppsala, Sweden, which are based on surface plasmon resonance (SPR) and permit monitoring of surface binding interactions in real time between analytes (molecules) of interest. Other biosensors can also be used.

Typical analytes that can be used in the context of the present disclosure include, but are not limited to, proteins and glycoproteins (e.g., antibodies or fragments thereof, affibodies, or aptamers), enzymes, receptors, antigens, haptens, peptides, chemical molecules (e.g. drug candidates or fragments thereof, specific or non-specific binders, chelators or aggregators), lipids, and carbohydrates.

The term “antibody” describes an immunoglobulin whether natural or partly or wholly synthetically produced. The antibody may be monoclonal or polyclonal and may be prepared by techniques that are well-known in the art such as immunization of a host and collection of sera (polyclonal), or by preparing continuous hybrid cell lines and collecting the secreted protein (monoclonal), or by cloning and expressing nucleotide sequences or mutagenized versions thereof, coding at least for the amino acid sequences required for specific binding of natural antibodies. The term “antibody” also covers any polypeptide or protein comprising an antibody antigen-binding site. Antibody fragments that comprise an antibody antigen-binding site include, but are not limited to molecules such as Fab, Fab′, Fab′-SH, scFv, Fv, dAb, Fd; and diabodies. Methods of generating sensing surfaces for use in biosensor systems are well known in the art. Multiple examples of such methods are set out, for example, in U.S. Pat. Nos. 5,492,840 and 5,242,828, the contents of which are incorporated herein by reference.

While in the detailed description and Examples that follow, the disclosed systems and methods are generally illustrated in the context of SPR spectroscopy, and more particularly the BIACORE® system, it is to be understood that the disclosed systems and methods are not limited to this detection method. Rather, any affinity-based detection method where interaction between analytes, and/or between analytes and a ligand, at a sensing surface can be detected may be employed, provided that a change at the sensing surface can be measured which is quantitatively indicative of binding or interaction.

The phenomenon of SPR is well known, suffice it to say that SPR arises when light is reflected under certain conditions at the interface between two media of different refractive indices, and the interface is coated by a metal film, typically silver or gold. In the BIACORE® instruments, the media are the sample solution and the glass of a sensor chip, which is contacted with the sample by a micro fluidic flow system. The metal film is a thin layer of gold on the chip surface. SPR causes a reduction in the intensity of the reflected light at a specific angle of reflection. This angle of minimum reflected light intensity varies with the refractive index close to the surface on the side opposite from the reflected light, which in the BIACORE® system is the sample side.

A schematic illustration of an SPR analysis, such as takes place in the BIACORE® system, is shown in FIG. 1. Sensor chip 101 has a gold film 102 supporting capturing molecules (ligands) 103, e.g. antibodies. A first sample solution 105 flows over the sensor surface, exposing the sensor surface (and the ligands 103 thereon) to analyte 104, e.g. an antigen, comprised in the first sample solution 105. In the context of FIG. 1, and the present disclosure more generally, the phrase “flow over” means that the sample solution flows past the sensor surface and contacts the sensor surface in so doing, enabling any analytes in the sample solution to interact with a suitable capturing molecule at the sensor surface. The term “over” is thus not to be interpreted as requiring any absolute orientation. For example, as shown in FIG. 1, the flow channel 105 may in practice be underneath the sensor 101. The sensor (and flow path) may also be oriented at 90 degrees to that shown in FIG. 1, or at any other orientation.

Monochromatic p-polarised light 106 from a light source 107 (such as an LED) is coupled by a prism 108 to the glass/metal interface 109 where the light is totally reflected. The intensity of the reflected light beam 110 is detected by an optical detection unit 111 (such as a photodetector array). When molecules in a sample solution (such as analytes 104) bind to the capturing molecules (such as ligands 103) on the sensor chip surface, the concentration, and therefore the refractive index at the surface, changes and an SPR response is detected. Plotting the response against time during the course of an interaction will provide a quantitative measure of the progress of the interaction. Such a plot, or kinetic or binding curve (binding isotherm) is usually called a response signal or sensorgram, also sometimes referred to in the art as an ‘affinity trace’ or ‘affinogram’. In the BIACORE® system, the SPR response values are expressed in resonance units (RU). One RU represents a change of 0.0001° in the angle of minimum reflected light intensity, which for most proteins and other biomolecules corresponds to a change in concentration of about 1 pg/mm² on the sensor surface. As sample containing an analyte contacts the sensor surface, the capturing molecule bound to the sensor surface interacts with the analyte in a step referred to as “association.” This step is indicated on the sensorgram by an increase in RU as the sample is initially brought into contact with the sensor surface. Conversely, “dissociation” normally occurs when the sample solution is replaced by, for example, a running buffer flow, at which point the analyte is removed from the surface. This step is indicated on the sensorgram by a drop in RU over time as analyte dissociates from the surface-bound ligand.

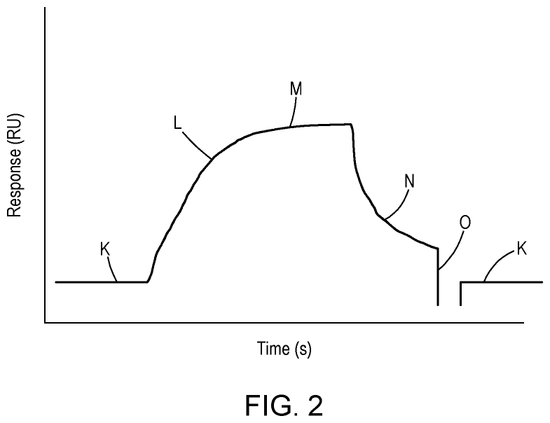

A representative sensorgram (binding curve) for a reversible interaction at the sensor chip surface is presented in FIG. 2. The binding curves produced by biosensor systems based on other detection principles mentioned above will have a similar appearance. In this example, the sensing surface has an immobilised capturing molecule (e.g. ligand) on it, for example an antibody. This interacts with a binding partner (analyte) in a sample solution as the solution flows over the sensor surface.

The vertical axis (y-axis) indicates the response (here in resonance units, RU) and the horizontal axis (x-axis) indicates the time (here in seconds). Initially, running buffer is passed continuously over the sensing surface giving the baseline response K in the sensorgram. A sample solution containing an analyte of interest is then injected. During sample injection, an increase in signal is observed due to binding of the analyte to the ligand at the sensor surface. This part L of the binding curve is usually referred to as the “association phase”. Eventually, a steady state condition is reached at or near the end of the association phase where the resonance signal plateaus at M (this state may, however, not always be achieved). It is to be noted that herein the term “steady state” is used synonymously with the term “equilibrium” (in other contexts the term “equilibrium” may be reserved to describe the ideal interaction model, since in practice binding could be constant over time even if a system is not in equilibrium). At the end of sample injection, the sample is replaced again with a continuous flow of running buffer and a decrease in signal reflects the dissociation, or release, of analyte from the sensor surface. This part N of the binding curve is usually referred to as the “dissociation phase”. The analysis is typically ended by a regeneration step where a solution capable of removing bound analyte from the surface, while (ideally) maintaining the activity of the ligand, is injected over the sensor surface. This is indicated in part O of the sensorgram. However, regeneration can be avoided where dissociation is already complete or is expected to become completed before the next analysis, which has the advantage of enhancing ligand preservation and reducing the number of operations. Injection of continuous running buffer restores the baseline K and the surface is now ready for a new analysis.

From the profiles of the association and dissociation phases L and N, respectively, information regarding the binding and dissociation kinetics is obtained, and the height of the resonance signal at M represents affinity (the response resulting from an interaction being related to the change in mass concentration on the surface). A detailed explanation of methods used to determine parameters such as surface binding rates, kinetic rate constants ka and kd, affinity constants (association constant KA and dissociation constant K_{D}), and other parameters, is disclosed in US2012/0244637 A1. Software for the analysis of kinetic and affinity data is commercially available. Thus, for example, evaluation of kinetic and affinity data produced by the BIACORE instruments is usually performed with the dedicated BIACORE insight evaluation software (supplied by Cytiva, Uppsala, Sweden) using numerical integration to calculate the differential rate equations and non-linear regression to fit the kinetic and affinity parameters by finding values for the variables that give the closest fit, reducing the sum of squared residuals to a minimum. A detailed discussion of the technical aspects of the BIACORE® instruments and the phenomenon of SPR may further be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE® instruments may be found in U.S. Pat. No. 5,492,840. The above publications as well as any other publications, patent applications, patents, or other references mentioned in this disclosure are incorporated by reference in their entirety.

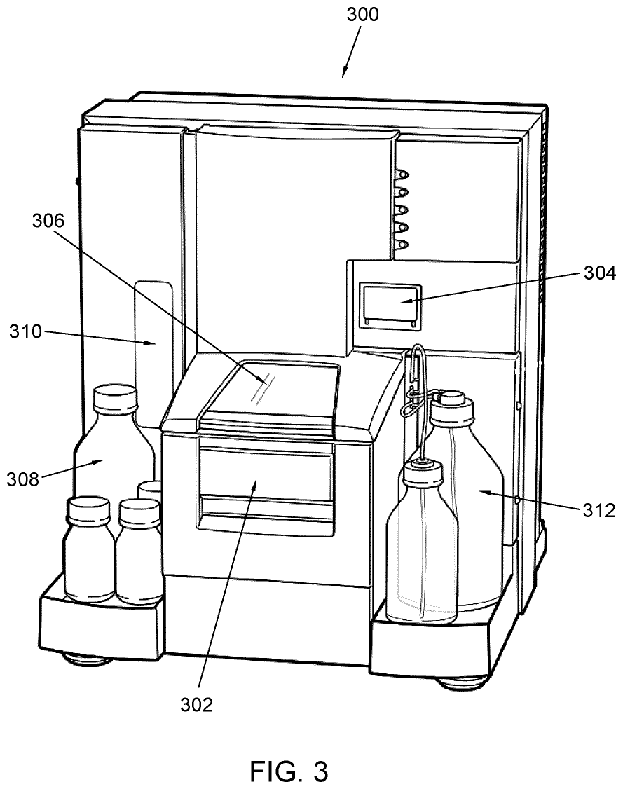

FIG. 3 shows a schematic illustration of an example sample analysis system (also referred to as a biosensor system) which can be used to implement the disclosed methods. In the example shown, the analysis system 300 is a BIACORE® T200 instrument, produced by Cytiva. It will be appreciated that this is a non-limiting example of a biosensor system and that the components described below can be generalised to other forms of biosensor systems. More generally, the disclosed methods can be incorporated in and applied to a wide array of other biosensor systems that operate under similar principles and are not limited to implementation in a BIACORE® system.

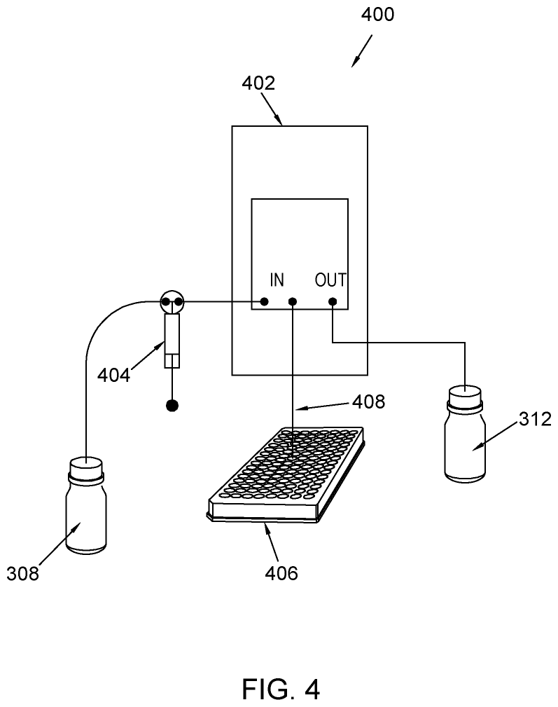

The analysis system 300 comprises a sample compartment 302, which is shown in the closed position in FIG. 3. The sample compartment 302 can be opened, and sample solution (generally held within one or more sample reservoirs as shown more clearly in FIG. 4) can be inserted into the compartment 302. Once sealed, viewing window 306 allows the sample solutions within compartment 302 to be viewed from outside. During analysis, a sample delivery system (also referred to as a fluid handling system) delivers sample solution from the sample reservoirs held within sample compartment 302 to a sensor surface, as described more fully below. At its simplest, the sample delivery system comprises a network of at least one pump and flow channel for routing sample and running buffer through the analysis system 300 and to the sensor. The sensor can be inserted and removed before/after analysis via a sensor chip port 304. Continuous running buffer can be provided from buffer reservoir 308, via a buffer pump (not show) housed in buffer pump compartment 310. Waste material can be collected in waste reservoir 312.

FIG. 4 shows an example sample delivery system 400 such as may be found within a biosensor system used to implement the disclosed methods, for example the analysis system 300 of FIG. 3. The sample delivery system 400 comprises a fluidic cartridge 402 configured to receive sample solution from sample solution reservoirs (e.g. sample vials or microplate wells) held in a sample array 406. In the example shown, the sample solution is extracted from a respective reservoir (e.g. vial) using a needle 408, which can aspirate and then inject sample into the fluidic cartridge. Other mechanisms for sample delivery will be apparent. The fluidic cartridge 402 is also configured to receive running buffer from running buffer reservoir 308. Flow of running buffer is controlled using a running buffer pump 404. Once sample solution or running buffer is received at fluidic cartridge 402, it can be provided to a sensor via one or more flow channels (not shown), as described in more detail below. Delivery of sample solution to the fluidic cartridge and/or sensor surface is preferably performed under action of a sample delivery pump (not shown) which is different to running buffer pump 404. This permits separate control of sample delivery and running buffer delivery. Alternatively, a single pump may switch between pulling running buffer from reservoir 308 and pulling sample solution from sample array 406 as required. Waste solution can be expelled from the sensor surface and flow channels, via fluidic cartridge 402 to waste reservoir 312.

Turning now to FIGS. 5 and 6, it is often desirable to analyse the behaviour of multiple analytes in succession when using a sample analysis system. For instance, first sample A containing analyte A′ is injected. Analyte A′ binds to the ligand on the sensor surface and then dissociates upon the introduction of running buffer flow. Subsequently, this process is repeated for samples B, C and D containing respective analytes B′, C′ and D′. The response curves associated with such an approach are shown schematically (and with simplified profiles) in FIG. 5. This approach allows the binding behaviour of analytes A′-D′ to the sensor surface be compared. For example, FIG. 5 shows a strong binding signal from analytes A′ and D′ and weaker binding signals from analytes B′ and C′.

However, the present inventors have identified that it would also be useful to determine how analytes A′-D′ interact with one another, and/or interact with a ligand on the sensor surface when there is more than one analyte present. Such analysis may have particular application in the study of a variety of analytes and ligands and permits new and insightful assays to be conducted, as described in the Implementations and Examples below. However, it will be appreciated that conducting such analysis is difficult when using the approach described above and shown in FIG. 5, because a first analyte will be removed, or at least significantly disturbed, by the introduction of running buffer flow between samples. The presently disclosed systems and methods seek to address this shortcoming, as will now be described.

In order to effectively analyse how first and second (and potentially more) analytes interact with one another, the present disclosure involves introducing first and second sample solutions (comprising the first and second analytes respectively) in sequence, one after another, with no intermediate running buffer flow. The present inventors have identified that, surprisingly, many assays can be performed effectively in this manner without a need for running buffer flow between each sample injection. Indeed, many assays and experiments benefit from the removal of running buffer flow between samples, as explained more fully below and evidenced by the Implementations and Examples outlined later.

The process of sample injection without intermediate running buffer flow can be performed for a plurality (i.e. two or more) sample solutions and is referred to herein as a ‘polyinjection’ process as noted above. To return to the above example of samples A-D, a polyinjection process involves introducing each sample in turn to the sensor surface, without any intermediate running buffer flow. The response associated with such an approach is shown schematically and in simplified form in FIG. 6. Polyinjection is particularly useful in that it allows interactions between analytes and ligands to be studied more effectively. In particular, molecules which are sensitive to buffer flow can be studied without fear of interreference by running buffer. Fast interactions involving successively injected analytes can also be studied more effectively because there is no ‘down-time’ between sample injections caused by the flow of running buffer between sample injections.

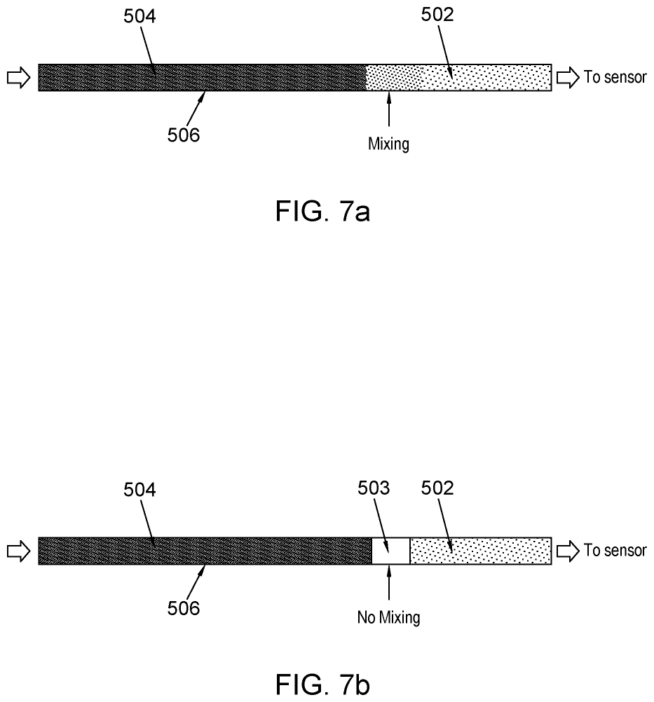

The present inventors have identified that polyinjection procedures can be made more effective through introduction of a gas segment between successive samples in the flow channel. In particular, if multiple samples are injected into the same flow channel for delivery to the sensor surface, then they will start to mix as they flow through the channel, as shown in FIG. 7a. This figure shows a first sample solution 502 comprising a first analyte and a second sample solution 504 comprising a second analyte flowing through a first channel 506. At the interface of the two samples, mixing occurs. Once mixing begins, the analytes contained in the first and second samples will begin to react within the first channel 506, i.e. before the samples reach the sensor surface. By the time the samples reach the sensor, the interaction or reaction between the analytes may be partially or fully over. Important reaction dynamics and characteristics may therefore be missed because they have taken place before the sensor surface has been reached and so cannot be analysed.

To address this problem, a gas volume or gas segment may be introduced in between sequential sample solutions. This gas volume effectively forms a bubble in the flow channel, separating the subsequent sample solutions and preventing mixing. This approach is shown in FIG. 7b, where first sample solution 502 is separated from second sample solution 504 by gas segment 503. This arrangement allows both samples to travel through the first channel 506 and reach the sensor without mixing occurring during transit. Only on reaching the sensor surface do the samples come into contact, at which point the interactions of the respective analytes can be analysed.

While the arrangement shown in FIG. 7b enables polyinjection to be performed without fear of premature mixing, the present inventors have further identified that allowing gas such as gas segment 503 to contact the sensor surface is undesirable. In particular, gas segment 503 may disturb the analyte from first sample 502 which has been deposited at the sensor surface. Gas segment 503 may even cause damage to the sensor surface in some instances.

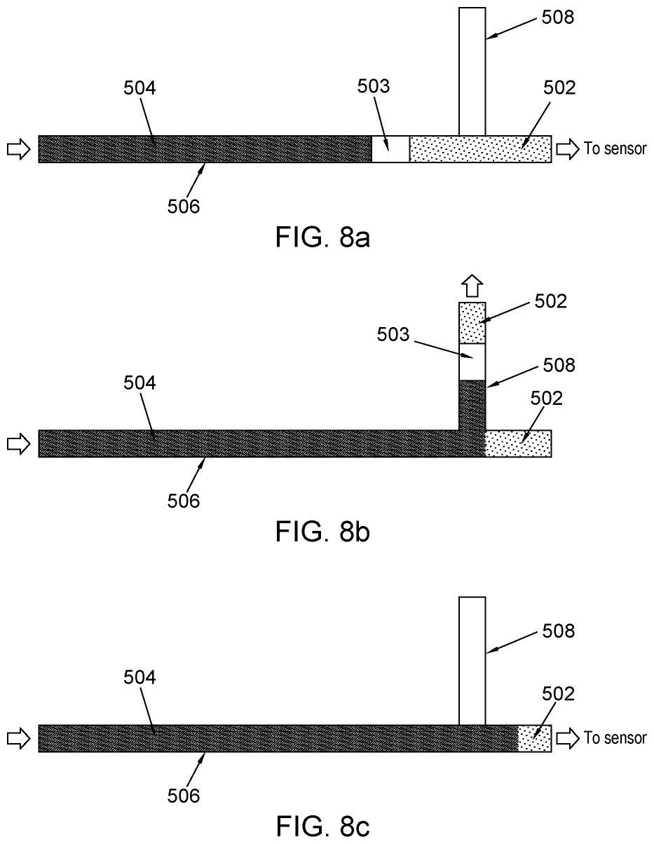

To address this, a second channel 508 may be provided near the end of the first channel 506, shortly before the sensor surface. This arrangement is shown in FIGS. 8a-8c. As in the example of FIG. 7b, the process begins by introducing first and second sample solutions 502,504 into the first channel 506, the samples being separated from one another by a gas segment 503. The first sample solution 502, second sample solution 504 and gas segment 503 are then flowed through the first channel 506 towards the sensor surface. In contrast to the FIG. 7b arrangement, however, prior to the gas segment 503 reaching the sensor surface, the gas segment 503 is extracted from the first channel 506 via a second channel 508. This is shown in FIG. 8b and ensures that the gas segment 503 does not contact the sensor surface. Second channel 508 may be routed directly to a waste reservoir such as reservoir 312 of FIG. 3.

Once the gas segment 503 has been extracted, the samples can be allowed to flow sequentially over the sensor surface as shown in FIG. 8c. The behaviour of the samples at the sensor surface can then be analysed. In particular, binding of analyte in at least one of the first sample 502 and second sample 504 can be analysed. This binding may be between analytes in the two samples (i.e. one of the analytes may effectively act as a ligand by binding to the sensor surface and then capturing the second analyte), and/or between the analytes and a ligand that has been previously immobilised on the sensor surface. Alternatively, only the first sample solution 502 may comprise an analyte and injection of the second sample solution 504 can be used to change the environment (e.g. pH) in which binding takes place, such that the behaviour of the analyte in these new conditions can be studied.

Because second channel 508 is positioned near the end of first channel 506, where fluid reaches the sensor surface, there is only minimal mixing between the first and second samples before the sensor surface is reached. Preferably, the gas segment 503 is removed no more than 40 seconds before the second sample 504 reaches the sensor surface. This time threshold ensures that the first and second sample solutions are not in contact in the first channel 506 long enough for significant mixing or interaction to occur. All or substantially all of the interaction between the sample solutions therefore takes place at the sensor surface and can thus be analysed. For fast-dissociating analytes, the time between the gas segment 503 being removed may be no more than 30 seconds, no more than 20 seconds, no more than 10 seconds, or no more than 1 second before the second sample 504 reaches the sensor surface. The selected time will depend on the analytes in question and can be determined by how much length of the first flow channel 506 is provided between the second flow channel 508 and the sensor surface.

Also with reference to FIGS. 5 and 6, it is often desirable to analyse the behaviour of multiple analytes in succession when using a sample analysis system. For instance, a first set of samples A, B, C and D may be analysed. First sample A containing analyte A′ is injected. Analyte A′ binds to the ligand on the sensor surface. The injection is followed by a flow of running buffer, during which some or all of analyte A′ may dissociate depending on the nature of the analyte. Subsequently, this process is repeated for samples B, C and D containing respective analytes B′, C′ and D′. The response signals 5502, 5504, 5506, 5508 generated by binding of analytes A′-D′ are shown in FIG. 5. In combination, these individual response signals provide the total binding signal 5500 for the assay of four sample solutions. Note that the response signals are shown schematically and with simplified profiles. In reality, each binding response signal 5502-5508 would resemble the response curve shown in FIG. 2 and discussed above. The approach shown in FIG. 5 allows the binding behaviour of analytes A′-D′ to the sensor surface be compared. For example, FIG. 5 shows a strong binding signal from analytes A′ and D′ and weaker binding signals from analytes B′ and C′.

In one implementation of FIG. 5, running buffer is passed over the sensor between sample injections to keep the system suitably wetted between samples. In other implementations, it may be preferable to omit this running buffer, however. In particular, the present inventors have identified that this may be desirable, for example, when analysing fast reactions where there is insufficient time for a running buffer step between analyte injections, or where fragile complexes need to be built up on the sensor surface which might be disturbed by running buffer flow. In these cases, as well as others, the present inventors have identified that it can be beneficial to inject samples sequentially one after another with no intermediate running buffer flow. The process of injecting a plurality (i.e. two or more) sample solutions without intermediate running buffer flow is referred to herein as a ‘polyinjection’ process. To return to the above example of set of sample solutions A-D, a polyinjection process involves introducing each sample in turn to the sensor surface, without any intermediate running buffer flow. The response associated with such an approach is shown schematically and in simplified form in FIG. 6. Here, individual response signals 6602, 6604, 6606, and 6608 again combine to give a total overall binding signal 6600 for the assay.

As mentioned, polyinjection of the sort shown in FIG. 6 is particularly useful in that it allows various interactions to be studied more effectively. In particular, molecules which are sensitive to buffer flow can be studied without fear of interference by running buffer. Fast interactions involving successively injected analytes can also be studied more effectively because there is no ‘down-time’ between sample injections caused by the flow of running buffer between sample injections.

Regardless of the method by which a set of samples is injected into the system, the result is an aggregate or total binding signal (or binding curve) for the set of samples, as shown in FIGS. 5 and 6. This binding signal is representative (i.e. comprises a combination of) of the binding response signals of each individual sample solution in the set of sample solutions.

A problem arises when analysing a set of sample solutions, however, in that existing methodologies make it difficult to determine which binding responses are dependent on or impacted by which other binding responses. It can also be difficult to determine whether a binding response of one sample is masked by a larger binding response of another sample. Consider, for instance, the binding signal 6600 of FIG. 6. This signal is comprised of four sub-signals 6602, 6604, 6606, 6608, produced by samples A-D (containing analytes A′-D′) respectively. From binding signal 6600, it is possible to determine that there is binding of analyte from each of samples A-D at the sensor surface. It is also possible to determine that analyte A′ has the largest binding response, while analyte C′ has the smallest binding response. However, more sophisticated analysis is difficult because it is hard to know how the binding signals 6602-6608 relate to or interfere with one another. For example, it cannot be accurately determined whether each of analytes B′, C′ and D′ is binding to the ligand or to another analyte. It also cannot be determined accurately whether the dissociation signal of one analyte is masking the association signal of a subsequently injected analyte, such that the true characteristics of that analyte's association are impossible to determine.

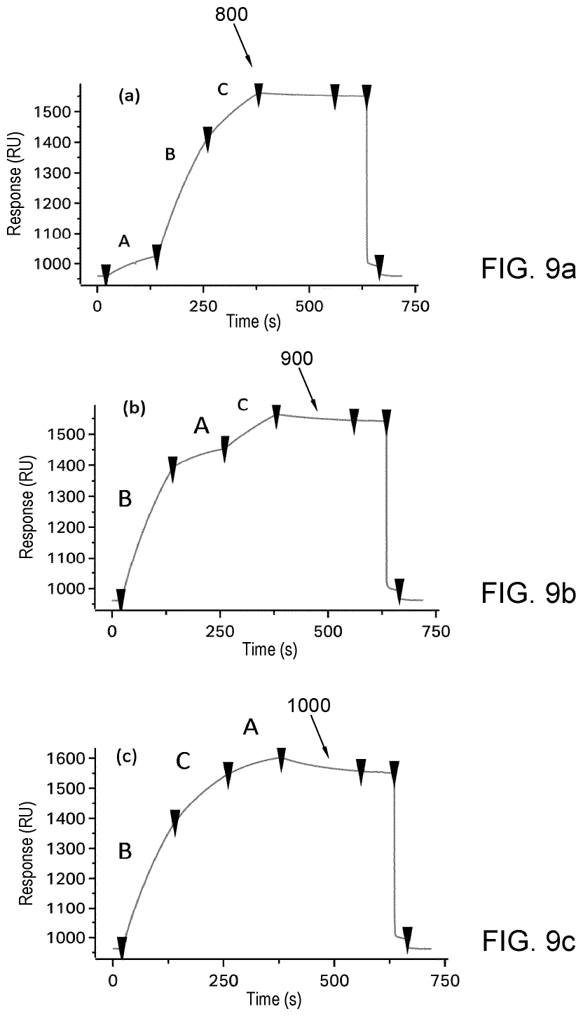

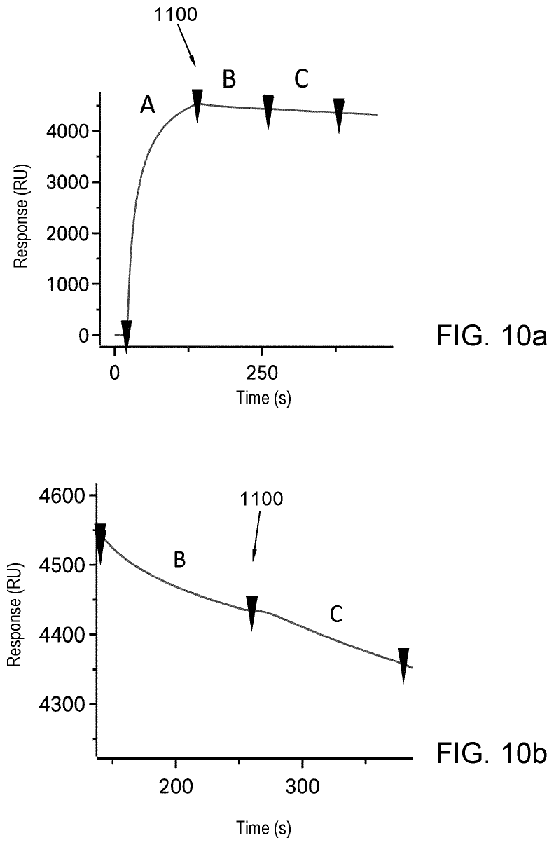

These problems may become more apparent through consideration of FIGS. 9 and 10 which demonstrate in more detail, and in relation to more realistic binding signals, how existing methodologies allow only limited analysis of combined binding signals. Once these shortcomings have been discussed, improved methodologies which overcome the discussed problems will also be outlined in this disclosure.

Turning first to FIGS. 9a-c, these Figures show three binding signals 800, 900 and 1000. In this example, the binding signals 800,900,1000 are generated by injection of a set of sample solutions. On injection, analyte arrives at the sensor surface and binds there, creating an association response signal. Then, as the sample begins to be replaced by the subsequently injected sample, the initial analyte begins to at least partially dissociate, generating a dissociation signal. The newly arriving analyte may begin to bind to the ligand, the analyte already present at the sensor surface, or neither. If there is binding, the new analyte generates an association signal which coincides with the dissociation signal of the first analyte.

In each assay shown in FIGS. 9a-c, the order in which the samples is injected is altered, as shown. In FIG. 9a, the sequence is A, B, C. In FIG. 9b, the sequence is B, A, C. This generates a different response signal 900. Finally, in FIG. 9c, the sequence is B, C, A. This generates yet another response signal 1000. Due to the change of order, each binding signal has a slightly different profile. Binding is exhibited by all three samples regardless of the order of injection.

From a consideration of FIGS. 9a-c, it becomes clear that the binding signals 800-1000 only provide limited information. For example, we know from these signals that binding occurs in respect of each analyte regardless of the order of injection. While that information is useful as a starting point, it would be advantageous to have more detailed data on the precise response signals generated by each sample.

Unfortunately, when using existing methodologies it is not possible to obtain this information from signals 800-1000 because the responses of the respective samples obscure one another. In particular, in FIG. 9a, the dissociation signal of A coincides with the association signal of B. This “mixing” of signals therefore means that the true association signal associated with sample B is obscured and impossible to determine accurately. Similarly, the dissociation signal of B coincides and therefore partially masks the association signal of C. This means that it is difficult to obtain an accurate understanding of the association (or indeed dissociation) signal of any of the samples. While in some cases it is possible to obtain this data by performing assays with only a single sample, in many cases that will not be possible for example where binding of one analyte (e.g. B′) is dependent on the presence of another analyte (e.g. A′). It is also impossible to ascertain from signals 800,900,100 whether the analytes A′, B′ and C′ compete for binding sites.

Turning next to FIGS. 10a-b, another limitation of existing methodologies becomes apparent. FIG. 10a shows a new binding response curve 1100 generated by the successive injection of a different set of sample solutions. For simplicity, these are again labelled A, B and C and contain respective analytes A′, B′ and C′, though it will be apparent that samples A, B and C in the example of FIG. 10 are different to samples A, B and C in FIG. 9. In the present example of FIG. 10, the assay involves injection of a polyclonal antibody in sample A at a high concentration of 670 nM. Sample B contains another antibody at a lower concentration of 10 nM, while sample C contains a protein with a molecular weight of 25 kDa (100 nM), i.e., approx. 6 times smaller than the antibodies injected in the first two samples. Such an assay may be carried out, for example, to analyse antibody-protein binding.

As can be seen, sample A generates a very large binding response, indicating that analyte A′ binds strongly to the ligand (or other capturing molecule) at the sensor surface. By contrast, there appears to be little or no response resulting from the injection of samples B and C. Even a closeup of the graph, shown in FIG. 10b, reveals little-while there is a slight ‘bump’ in the response on injection of B and C, the general trend is still one of dissociation i.e. reduction in signal. This is because the dissociation response of sample A has obscured or “swamped” the respective association responses of samples B and C. This behaviour makes it very difficult to ascertain any meaningful information about the behaviour of samples B and C, because they are so overwhelmed by the response of A. A signal of the sort shown in FIGS. 10a-b may occur, for example, when there is a large discrepancy in molar ratios between the samples being analysed. In such a scenario existing analysis techniques provide only limited information and ability to analyse sample behaviour.

As can be seen from the above explanation and consideration of FIGS. 9 and 10, existing sample analysis methodologies leave much to be desired when analysing the behaviour of successively injected samples. The present inventors have identified a new advantageous methodology which addresses these shortcomings and allows more sophisticated and effective analysis of a variety of analyte binding interactions.

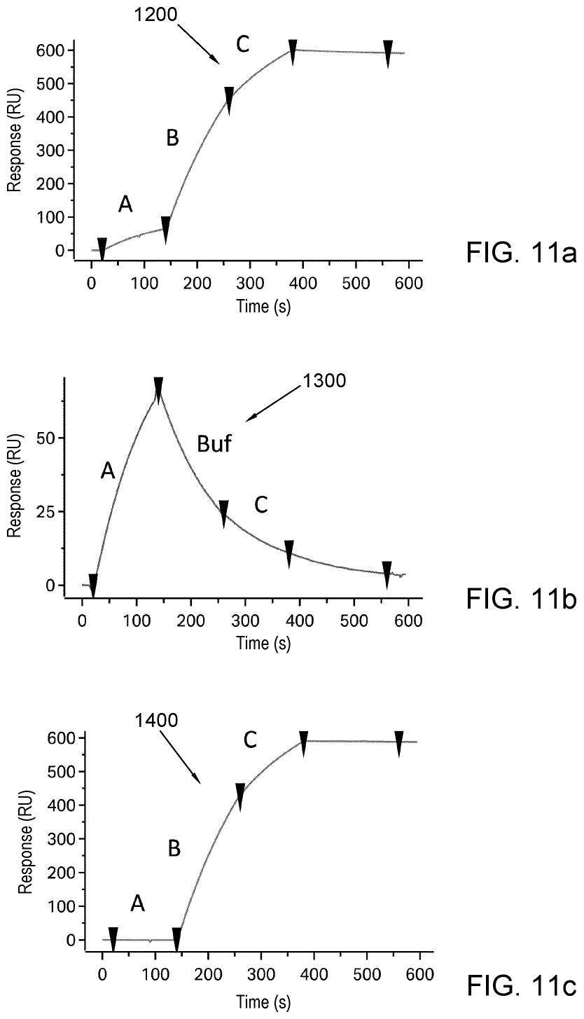

A realisation underpinning the improved methodologies of the present disclosure is the fact that a greater variety of sample characteristics can be determined through use of successive assays and subtraction of the resulting binding signals. This approach is referred to as “cycle subtraction” in the present disclosure. An example implementation of the cycle subtraction methodology will now be described with reference to FIGS. 11-17.

Turning first to FIGS. 11a-c, binding response curves implementing a cycle subtraction procedure are shown. FIG. 11a shows a first binding signal 1200 resulting from the subsequent injection of three samples, A, B and C as part of a polyinjection assay.

The present inventors have discovered that running a second assay with one of the samples replaced with a buffer sample can enable greater insight and more effective analysis of analyte behaviour. As noted above, a buffer sample solution is to be contrasted with continuous flow of running buffer. In particular, a buffer sample solution (or sample of buffer) comprises a specified volume of buffer provided via a sample injection pump or similar sample injection mechanism, typically from a sample vial, in the same way as any other sample solution. A buffer sample is injected in the context of analysing analyte behaviour at the sensor surface. This is in contrast to a flow of running buffer which is provided from a buffer reservoir, by a buffer reservoir pump, and which has the sole purpose of ‘flushing out’ and wetting the system before or after an assay has been performed.

The second assay in the present example involves subsequent injection of Sample A, a buffer sample, and sample C. Hence, the second assay is the same as the first assay of FIG. 11a, with the exception that sample B has been replaced with a buffer sample, as shown in Table 1 below. The buffer sample exhibits no response at the sensor surface because there is no analyte contained therein.

TABLE 1
Samples involved in the first and second assays resulting
in binding signals 1200 and 1300 respectively.

| | Sample | Sample | Sample | Resultant |
|---|---|---|---|---|
| Assay | A | B | C | binding signal |
| 1 | A′ | B′ | C′ | 1200 (FIG. 11a) |
| 2 | A′ | Buffer | C′ | 1300 (FIG. 11b) |
| | | sample | | |

The second assay results in a second binding signal 1300, shown in FIG. 11b. By subtracting the second response signal 1300 of FIG. 11b from the first response signal 1200 of FIG. 11a, greater insight into binding behaviours of the analytes can be obtained. The resulting binding signal 1400, which can be considered a third or difference signal, is shown in FIG. 11c. From this difference signal 1400, at least two features of the binding response can be analysed in a manner that was not possible from the original binding signal 1200 of FIG. 11a. Firstly, the true form and magnitude of the response caused by sample B has now been revealed. In the present case, the magnitude of the response in FIG. 11a, can now be determined more accurately because any obscuring signal caused by the association and dissociation of A has been removed. Secondly, we can see binding response of both sample B and C in difference signal 1400. This indicates that samples B and C are binding dependent. If sample B possessed an independency against sample C, only binding response of sample B alone would been displayed. However, FIG. 11c shows binding responses from both sample B and C. Put more precisely, the difference signal 1400 shows us that analyte C′ will only bind in the presence of analyte B′ This binding dependency was not apparent from the original signal 1200 but has been revealed by the cycle subtraction methodology of the present disclosure.

Hence, it can be seen that the disclosed methodology enables not only more accurate analysis of particular binding responses (here the signal of sample B), but also enables binding dependencies to be uncovered. More effective sample analysis is thus made possible.

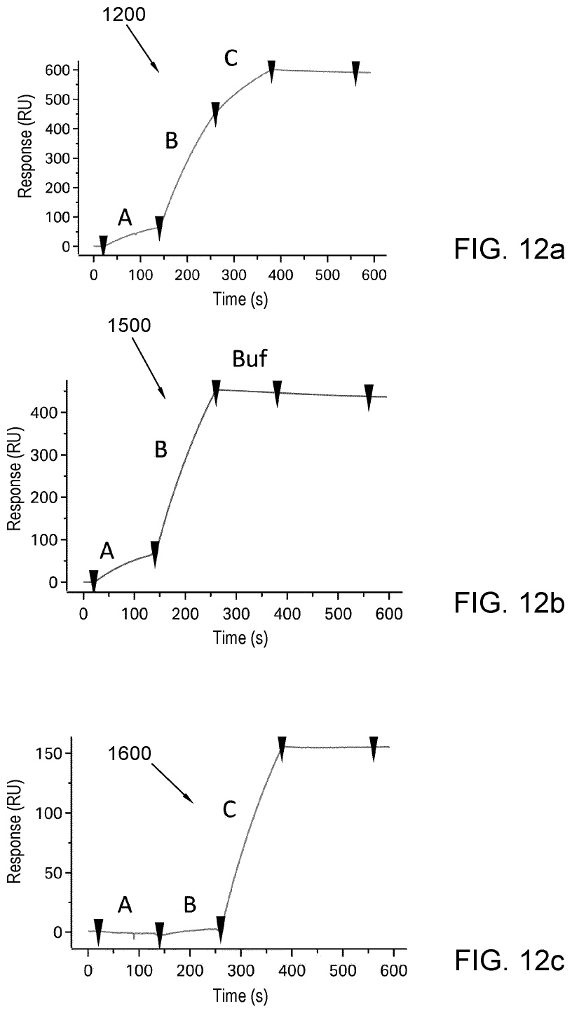

Another example implementation of the disclosed cycle subtraction methodology is shown in FIG. 12. FIG. 12a shows the same response signal 1200 as shown in FIG. 11a, caused by the successive injection of samples A, B and C.

FIG. 12b shows a new response signal 1500 resulting from a second assay. The second assay in this example involves subsequent injection of sample A, sample B, and a buffer sample. Hence, the second assay in this case is once again the same as the first assay with the exception that this time sample C has been replaced with a buffer sample. The buffer sample exhibits no response at the sensor surface because it does not contain any analyte. Response signal 1500 is thus similar to response signal 1300 of FIG. 11b, except that now sample C has been replaced with buffer as shown in Table 2:

TABLE 2
Samples involved in the first and second assays resulting
in binding signals 1200 and 1500 respectively

| | Sample | Sample | Sample | Resultant |
|---|---|---|---|---|
| Assay | A | B | C | binding signal |
| 1 | A′ | B′ | C′ | 1200 (FIG. 12a) |
| 2 | A′ | B′ | Buffer | 1500 (FIG. 12b) |
| | | | sample | |

FIG. 12c again shows a resulting binding signal 1600 generated through subtraction of the second binding signal 1500 from the first binding signal 1200. Analogously to FIG. 11c above, a primary benefit of computing this difference signal 1600 is that the binding response of sample C can now be more accurately revealed, because the obscuring association and dissociation signals of samples A and B have been removed. The response signal 1600 shows only a binding response of sample C. However, from binding signal 1400 we learnt that sample C only can bind in presence of sample B. The observed binding signal 1600 of sample C is thus not independent even though only binding of C is displayed in FIG. 1600.

It is important to note that, from FIG. 11c, we know that sample C is dependent on the presence of sample B. Hence, injection of sample C on its own would not have generated a signal. Thus, when using previous methodologies it would simply not have been possible to generate a binding signal of the sort show in FIG. 12c where the binding signal of sample C is accurately isolated from other signals and can be analysed independently. By contrast, the presently disclosed new methodologies make detailed analysis of the signal response of sample C possible.

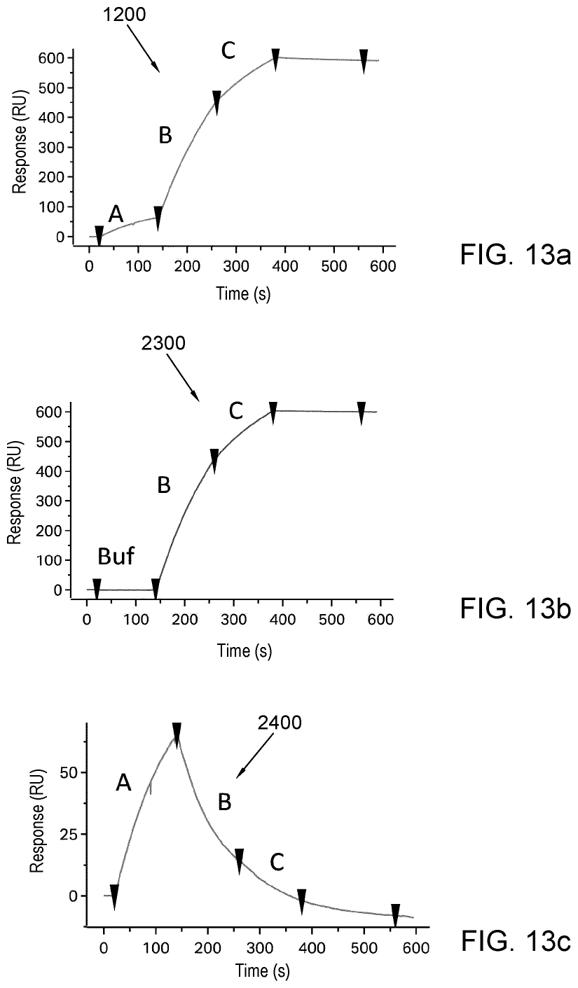

Another example implementation of the disclosed cycle subtraction methodology is shown in FIG. 13. FIG. 13a again shows the same response signal 1200 as shown in FIGS. 11a and 12a, caused by the successive injection of samples A, B and C.

FIG. 13b shows a new response signal 2300 resulting from a further subsequent assay. The subsequent assay in this example involves injection of a buffer sample, sample B, and sample C. Hence, the subsequent assay in this case is once again the same as the first assay with the exception that this time sample A has been replaced with a buffer sample. The buffer sample exhibits no response at the sensor surface because it does not contain any analyte. Response signal 2300 is thus similar to response signal 1300 of FIG. 11b and response signal 1500 of FIG. 12b, except that now it is sample A that has been replaced with buffer as shown in Table 3:

TABLE 3
Samples involved in the first and second assays resulting
in binding signals 1200 and 2300 respectively

| | Sample | Sample | Sample | Resultant |
|---|---|---|---|---|
| Assay | A | B | C | binding signal |
| 1 | A′ | B′ | C′ | 1200 (FIG. 13a) |
| 2 | Buffer | B′ | C′ | 2300 (FIG. 13b) |
| | sample | | | |

FIG. 13c again shows the resulting binding signal 2400 generated through subtraction of the second binding signal 2300 from the first binding signal 1200.

Analogously to FIGS. 11c and 12c above, a primary benefit of computing this difference signal 2400 is that the binding response of sample A (specifically its dissociation curve) can now be more accurately revealed, because the obscuring association and dissociation signals of sample B and obscuring association signal of sample C have been removed. The response of sample A replicates that which one would expect from an independent binding of an analyte in isolation (note similarity to response curve of FIG. 2). Note also that FIG. 13c replicates FIG. 11b (where there was buffer in sample B and sample C revealed no binding), again indicating that sample A is independent of the presence of sample B and sample C.

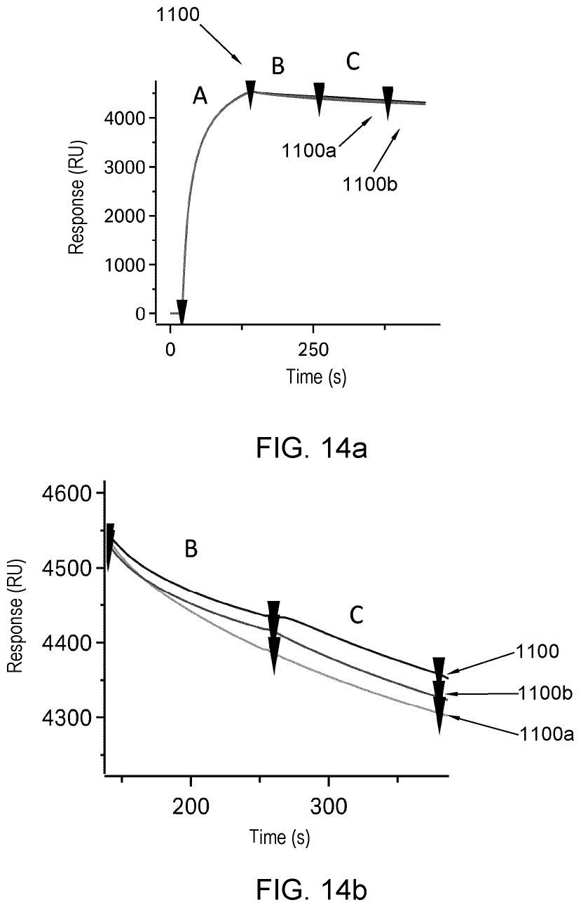

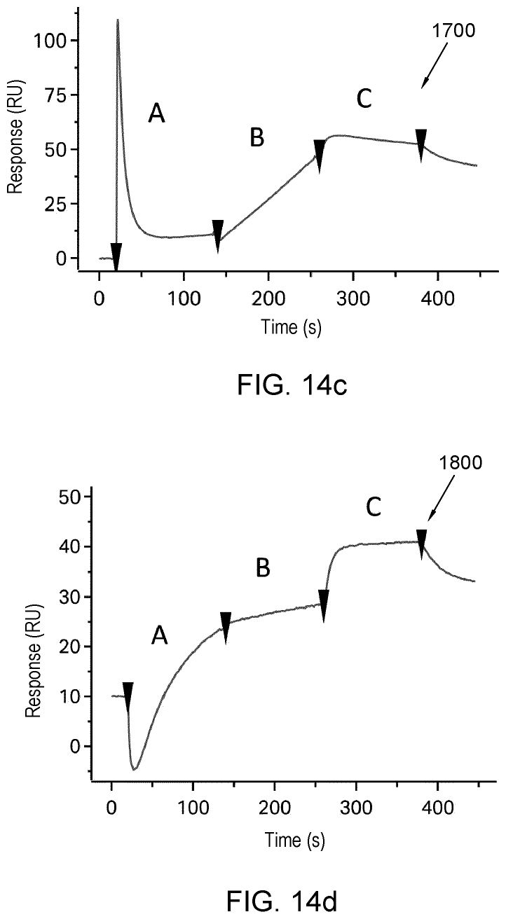

Turning now to a further example where the disclosed methodologies permit improved analysis, FIG. 14a shows the same binding response signal 1100 as shown in FIG. 10a. As noted previously, due to the large discrepancy in molar ratios, the response of sample A dwarfs that of samples B and C, such that the response of samples B and C are almost indecipherable. Additional insight into the behaviour of B and C can however be gained by use of the presently disclosed methodologies, namely by conducting a cycle subtraction in the manner outlined above.

To enable this, in the present example, two further assays are performed as shown in Table 4.

TABLE 4
Samples involved in the first, second and third assays resulting
in binding signals 1100, 1100a and 1100b respectively

| | Sample | Sample | Sample | Resultant |
|---|---|---|---|---|
| Assay | A | B | C | binding signal |
| 1 | A′ | B′ | C′ | 1100 (FIG. 14a/b) |
| 2 | A′ | Buffer | C′ | 1100a (FIG. 14a/b) |
| | | sample | | |
| 3 | A′ | B′ | Buffer | 1100b (FIG. 14a/b) |
| | | | sample | |

Firstly, the assay is repeated but with sample B replaced with a buffer sample. This generates binding signal 1100a. Then, the assay is repeated again but with sample C replaced with a buffer sample. This generates binding signal 1100b. Because these signals are so similar to one another and to original signal 1100, all three signals are shown on the same plot in FIG. 14a. FIG. 14b shows a closeup of the segment during which samples B and C are injected. FIG. 14b is therefore equivalent to FIG. 10b, except now binding signals for a second and third assay are shown also.

Now, by using cycle subtraction, the behaviour of samples B and C can be studied effectively, as shown in FIGS. 14c and 14d. Turning first to FIG. 14c, subtraction of binding signal 1100a (buffer sample as sample B) from original binding signal 1100 generates a first difference binding signal 1700. As can be seen, the obscuring “noise” from the dissociation of sample A is now largely removed, and the association signal caused by analyte B′ can be accurately determined. In particular, an upwards association curve that was previously masked by the downward dissociation signal of sample A can now be seen and analysed.

Similarly, FIG. 14d shows a second difference binding signal 1800 produced by subtracting binding signal 1100b (buffer sample as sample C) from original binding signal 1100. As in the case of FIG. 14c, the obscuring “noise” from the dissociation of sample A is now largely removed, and the association signal caused by analyte C′ can be accurately determined. In particular, an upwards association curve that was previously masked by the dissociation signal of A can now be seen and analysed.

Hence, it can be seen that the disclosed methodologies enable binding signals that were previously hidden to be revealed and analysed accurately.

Another important area where the disclosed methodologies provide benefits is in the process of “epitope binning”. This involves determining which epitopes (e.g. of an antigen) analytes (e.g. antibodies) bind to. During drug development and antibody screening, it is important to know how antibodies interact with antigens, particularly whether two or more antibodies compete for a single antigen epitope. This analysis is made significantly simpler by the disclosed methodologies, as will now be explained.

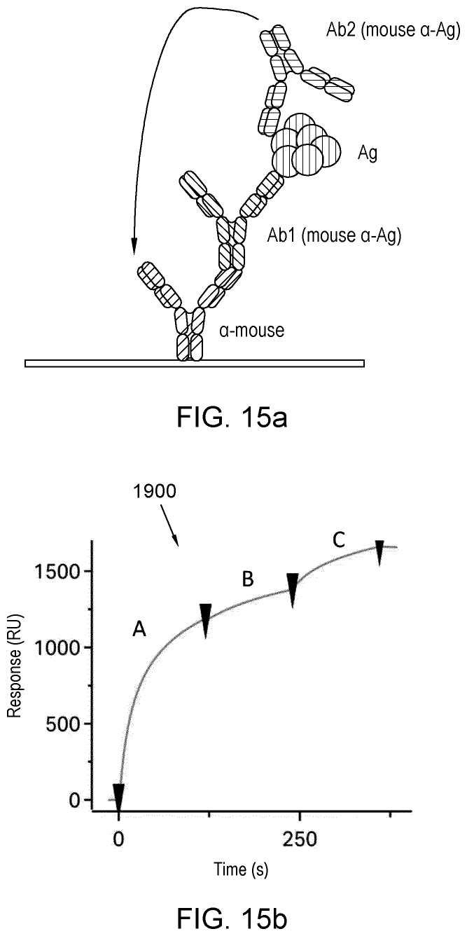

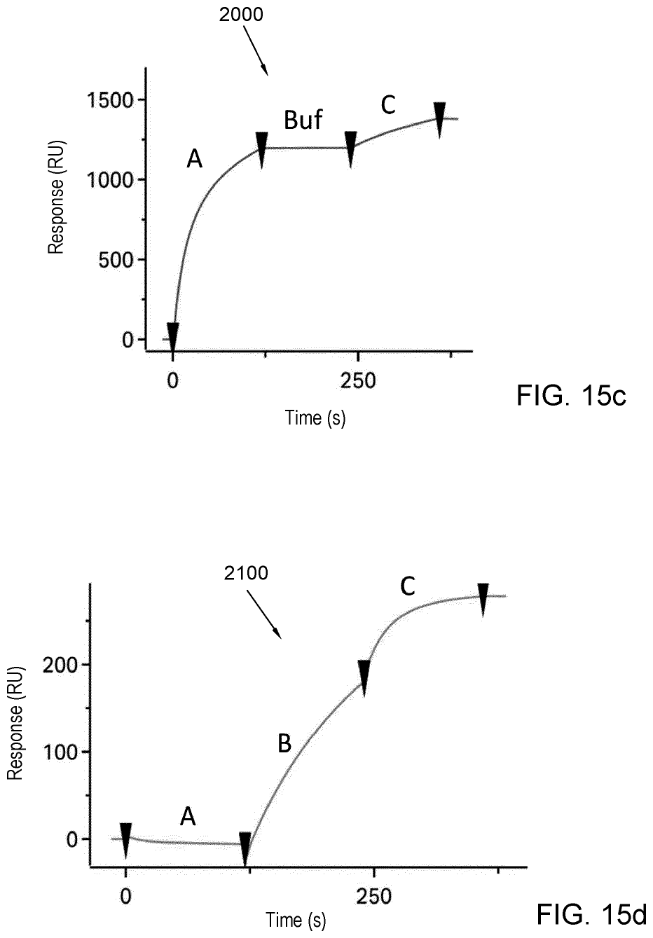

A first example of such an epitope binning analysis will initially be described with reference to FIG. 15. FIG. 15a shows an example binding complex that may be formed at the sensor surface during a particular assay. An anti-mouse antibody (a-mouse) is immobilized at a sensor surface to act as a ligand. Three samples, A, B and C are then successively injected in the manner described above. In this example the first sample solution A contains a first antibody of interest, Ab1. The second sample solution B contains an antigen (Ag). Finally, the third sample solution C contains a second antibody of interest, Ab2.

In many cases it is desirable to determine whether or not antibodies Ab1 and Ab2 bind to antigen Ag in the presence of one another. Such analysis can determine whether Ab2 and Ab1 compete for the same epitope (binding site) on the antigen, which can inform drug discovery and antibody screening investigations as noted above. Unfortunately, analysing this interaction using existing methodologies is difficult, because Ab2 in this example (and in many other ‘real-world’ examples) also binds to the ligand (a-mouse in this example). This second binding opportunity available to Ab2 is indicated by the arrow in FIG. 13a. Accordingly, when sample C containing Ab2 is injected there will be a binding response signal, but it will be impossible to know how much of this signal is caused by binding between Ab2 and the antigen Ag, and how much is caused by Ab2 binding to vacant binding sites at the a-mouse ligand. In other examples, binding between the two antibodies may occur and produce yet another “noise” signal that obscures the signal of interest between Ab2 and the antigen.

Traditionally, this problem is addressed by using so-called “blocking” molecules to block any vacant binding sites, for example sites at the ligand, before Ab2 is injected.

Once such blocking molecules are added, Ab2 has no binding sites at the ligand to bind to, and so there is no unwanted “noise” signal caused by such binding once Ab2 is injected. The magnitude of binding between Ab2 and the antigen Ag can then be accurately determined.

Unfortunately, however, developing an assay to include such a blocking molecule is time-consuming and difficult. Suitable blocking molecule candidates must be identified and tested. Often, blocking candidates exhibit poor performance and are not as effective as anticipated from theory, meaning that frequently a large number of candidates must be tested. In practice this can take several weeks or months even in sophisticated laboratory settings. This places a significant barrier on performing high volumes of assays because each assay must be preceded by an investigation to find a suitable blocking molecule.

The presently disclosed cycle subtraction methodology enables complexes of the sort shown in FIG. 15a to be analysed without requiring blocking molecules, as will now be described. This provides significant benefits and makes large scale analyses more feasible because the need to first identify a suitable blocking molecule is removed.

As in the implementations described above, the present example implementation begins by injecting a first set of sample solutions A, B and C which in this example contain analytes Ab1, Ag and Ab2 respectively. This causes a complex of the sort shown in FIG. 15a to form and generates a first binding signal 1900 as shown in FIG. 15b. As in the other implementations of the disclosed methodology, the assay is then repeated with a subset of the sample solutions and a buffer sample. In this example, the antigen Ag is replaced with a buffer sample in the second assay as shown in Table 5:

TABLE 5
Samples involved in the first and second assays resulting
in binding signals 1900 and 2000 respectively

| | Sample | Sample | Sample | Resultant |
|---|---|---|---|---|
| Assay | A | B | C | binding signal |
| 1 | Ab1 | Ag | Ab2 | 1900 (FIG. 15b) |
| 2 | Ab1 | Buffer | Ab2 | 2000 (FIG. 15c) |
| | | sample | | |

The second assay generates a second binding signal 2000, shown in FIG. 15c. As before, the disclosed methodology then involves subtracting binding signal 2000 from binding signal 1900. This cycle subtraction removes any ‘noise’ caused by binding between Ab2 and the ligand on the sensor surface (or indeed from binding between Ab2 and Ab1, if such binding occurs) to leave only the binding of interest, namely that between Ab2 and Ag. The resultant difference binding signal 2100 is shown in FIG. 13d. As can be seen, in this case, there is still a noticeable association signal for sample C even once any Ab2-ligand binding has been subtracted away. This shows that there is binding between Ab2 and the antigen Ag even in the presence of Ab1, meaning that Ab2 and Ab1 do not compete for the same epitope of Ag. Crucially, this useful insight has been obtained without requiring a blocking molecule, representing a significant improvement over existing methodologies for investigating epitope competition.

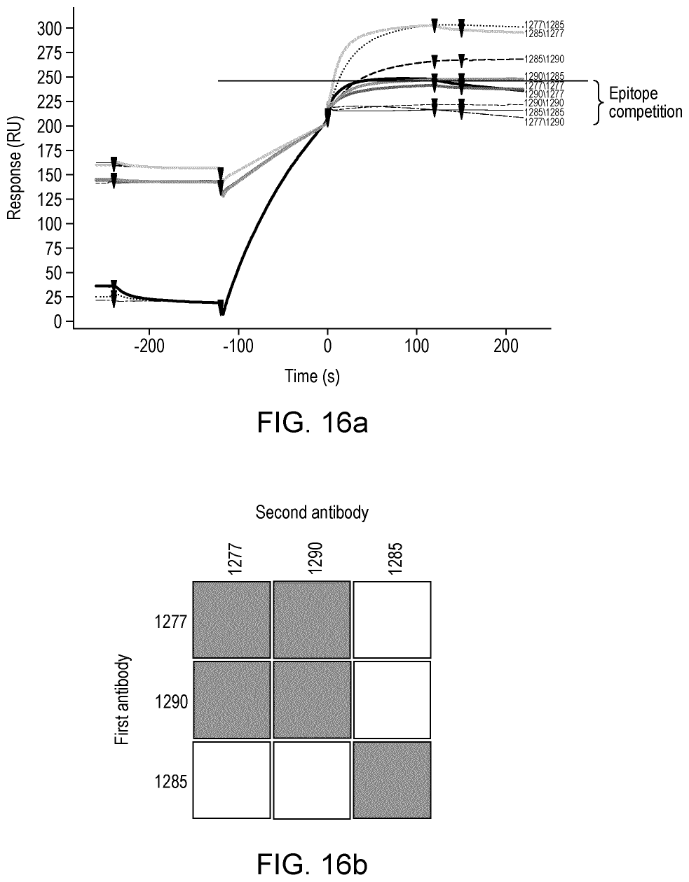

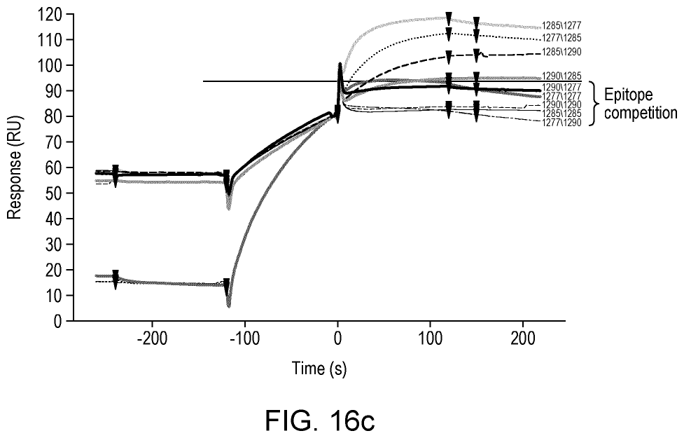

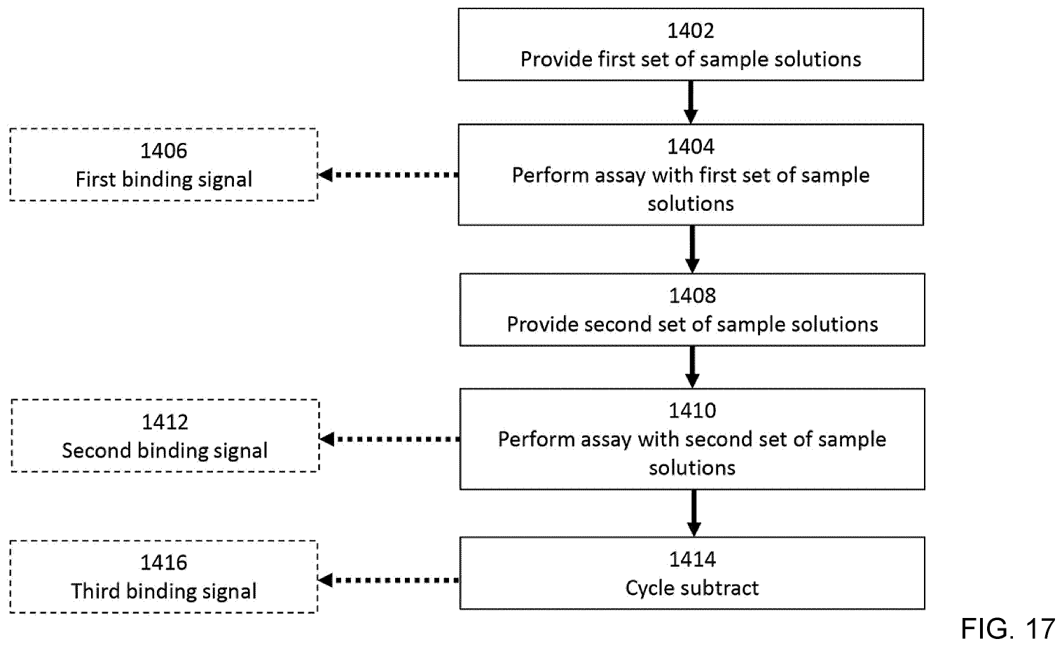

It will be appreciated that the examples described above are all deliberately simple to aid understanding. All the methodologies disclosed herein can be applied to more complex assays involving a greater variety of analytes of interest. One such example is shown in FIG. 16a which shows a number of difference response signals obtained through cycle subtraction in the manner described above. Specifically, these signals show the results from an epitope binning experiment that included three antibodies, labelled 1277, 1285 and 1290 respectively, and an antigen Human Creatin Kinase (CKMB). A more detailed example setup for conducting such an experiment is described in Example 2 below.

The antibodies were analysed in pairs, two at a time. Samples were injected and analysed, and the antigen was then replaced in a subsequent assay by a buffer sample in the same manner as described above with reference to FIGS. 11-15. This process was repeated with each antibody pair and with the antibodies injected in different orders, as shown in Table 6 below.

TABLE 6
Samples involved in eighteen assays performed
to investigate epitope competition in an epitope
binning analysis between three antibodies

| Assay | Sample A | Sample B | Sample C |
|---|---|---|---|
| 1 | 1277 | CKMB | 1277 |
| 2 | 1277 | Buffer sample | 1277 |
| 3 | 1277 | CKMB | 1285 |
| 4 | 1277 | Buffer sample | 1285 |
| 5 | 1277 | CKMB | 1290 |
| 6 | 1277 | Buffer sample | 1290 |
| 7 | 1285 | CKMB | 1285 |
| 8 | 1285 | Buffer sample | 1285 |
| 9 | 1285 | CKMB | 1277 |
| 10 | 1285 | Buffer sample | 1277 |
| 11 | 1285 | CKMB | 1290 |
| 12 | 1285 | Buffer sample | 1290 |
| 13 | 1290 | CKMB | 1290 |
| 14 | 1290 | Buffer sample | 1290 |
| 15 | 1290 | CKMB | 1285 |
| 16 | 1290 | Buffer sample | 1285 |
| 17 | 1290 | CKMB | 1277 |
| 18 | 1290 | Buffer sample | 1277 |

Binding signals obtained from even-numbered assays (involving a buffer sample) were subtracted from the binding signals of preceding odd-numbered assays via cycle subtraction as described above. For example, the binding signal generated by assay 2 was subtracted from the binding signal generated by assay 1. Assay 4 was subtracted from assay 3, and so on. This cycle subtraction generated nine difference binding signals, which are shown in FIG. 16a.

From these signals, it is possible to determine which antibodies compete for the same epitopes and how the ordering affects the competition. It is assumed that where the same antibody is injected twice, there will be epitope competition because an antibody naturally competes with itself. Hence, any difference signals below the highest self-competing signal (which in this example was 1277/1277) was deemed to be indicative of epitope competition. This “epitope competition” area of the graph is shown in FIG. 16a. As can be seen, all but four antibody combinations competed with one another. This result is also shown schematically in FIG. 16b, where greyed out segments represent epitope competition and white segments represent no competition. The left hand column shows the first antibody injected in the assay and the top row shows the second antibody injected. The table thus indicates that there is no competition between analytes 1285 and 1277, regardless of which order the antibodies are injected. The same is true of antibodies 1285 and 1290, although it is notable from the graph of FIG. 16a that the 1290/1285 difference curve is close to the 1277/1277 cut-off. This may indicate that further investigation into the 1290/1285 interaction may be warranted to determine why the binding response may be more muted when the antibodies are injected in this order. All other combinations and orders of antibodies in the present example result in epitope competition (i.e. fall at or below the 1277/1277 cut-off, in this example). It will be appreciated that in other examples antibodies may exhibit uni-directional competition, i.e. competition that is dependent on the order of injection.

It is to be emphasised that all of this information concerning epitope competition has and can be obtained solely by running assays using the three antibodies, antigen and buffer samples and conducting cycle subtraction in the manner disclosed above. No blocking molecules to block free ligand binding sites needed to be utilised, because any unwanted binding signal between antibodies and ligands were removed through the cycle subtraction methodology. Any number and combination of antibodies can be assayed and investigated in this manner. This presents a significant improvement over existing methodologies that rely on use of blocking molecules.

FIG. 16c shows results from a similar experiment, however in this case a lower ligand density was used on the surface (approx. 1000 RU compared with around 8700 RU in the analysis of FIG. 16a). As can be seen, the same results regarding which antibodies competed were replicated. The fact that the same results can be obtained regardless of ligand density shows the robustness of the present methodology.

The above-described cycle subtraction methodology for assaying multiple sets of sample solutions is summarised in FIG. 17. At block 1402, a first set of sample solutions comprising at least three sample solutions is provided. Typically, although not necessarily, the samples are aspirated and injected into a flow channel which is configured to deliver sample to a sensor surface. At block 1404, an assay is performed with the first set of sample solutions, during which each sample solution in the first set of sample solutions is flowed sequentially over the sensor surface. This generates a first binding signal 1406.

Next, a second set of sample solutions is provided at block 1408. The second set of sample solutions comprises a subset of the first set of sample solutions and a buffer sample solution. In other words, at least one sample solution of the first set of sample solutions provided at block 1402 has now been replaced by a buffer sample. At block 1410, an assay is performed with the second set of sample solutions, during which each sample solution in the second set of sample solutions is flowed sequentially over the sensor surface. This generates a second binding signal 1412.

Then, at block 1414, the second binding signal 1412 is subtracted from the first binding signal 1406 (“cycle subtraction”). This generates a third (difference) binding signal 1416.

Many of the above implementations, as well as the method of FIG. 17 generally, provide particular utility when using a polyinjection methodology, where samples are injected sequentially without intermediate running buffer or any “down time” between samples. In such implementations, the inventors have identified a number of additional optional features which can be used to improve the effectiveness of the assay.

One such feature relates to separation of subsequently injected samples using a gas segment. The present inventors have identified that polyinjection procedures can be made more effective through introduction of a gas segment between successive samples in the flow channel, as is described above in relation to FIGS. 7 and 8.

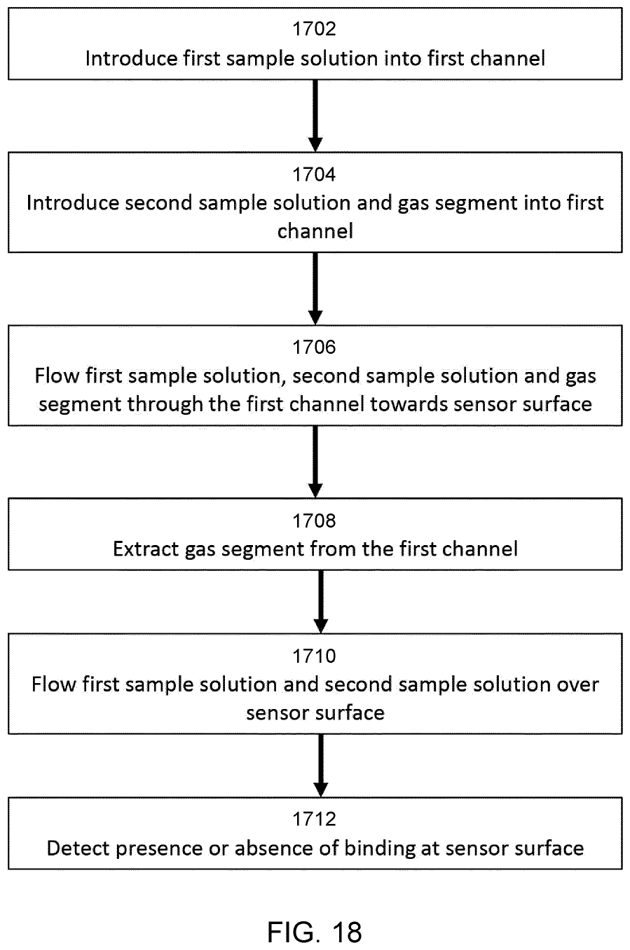

A method incorporating the above-described steps is shown in FIG. 18. The method involves provision and extraction of a gas segment, thought it will be appreciated that these steps may be omitted in some cases, particularly where the first flow channel is sufficiently short that mixing is negligible or where the interaction is such that the impact of mixing of the solutions prior to arrival at the sensor is minimal. In most cases, however, avoiding mixing in the first channel will enable more effective analysis of the sample interaction.

The method of FIG. 18 involves introducing, at block 1702, a first sample solution into a first channel, for example channel 506 of an analysis system 300. The first channel is configured to deliver sample solution to a sensor surface as shown in FIGS. 8a-c. At block 1704, a second sample solution is introduced into the first channel. As noted, this example implementation involves use of a gas segment.

Accordingly, the second sample solution is separated from the first sample solution in the first channel by a gas segment as described above with reference to FIGS. 7b and 8a. The gas segment prevents mixing of the samples in the first channel.

At block 1706, the first sample solution, second sample solution and gas segment are flowed through the first channel towards the sensor surface. This flowing is typically performed under action of a first pump of the analysis system which is configured to cause sample to flow through the first channel towards and over the sensor surface. Prior to the gas segment reaching the sensor surface, the gas segment is extracted from the first channel via a second channel at block 1708. This ensures that the gas segment does not contact the sensor surface. As noted above, block 1708 preferably happens at most 40 seconds prior to the second sample solution reaching the sensor surface, to ensure that any mixing and interaction between the first and second sample solutions in the flow channel (i.e. prior to reaching the sensor surface) is negligible for the purposes of analysis.

At block 1710, the first sample solution and the second sample solution are flowed over the sensor surface such that they may interact with one another and/or a ligand already provided at the sensor surface. It will be appreciated that while block 910 is shown after block 908 in FIG. 18, some of the first solution may already be present at the sensor surface when block 1708 (extraction of the gas segment) occurs. This is shown in FIG. 8b where first sample solution 502 has already partly contacted the sensor (not shown, but present to the right of the first flow channel 506 of FIG. 8b) when the gas segment 503 is extracted.

At block 1712, the interaction behaviour of the first and second samples at the sensor surface is analysed, in particular to detect the presence or absence of binding of analyte from at least one of the sample solutions at the sensor surface.

Advantageously, no running buffer solution (e.g. from running buffer reservoir 308 of FIG. 3) is passed over the sensor surface throughout the method of FIG. 18, i.e. until the interaction of the sample solutions has completed to a sufficient degree where the desired analysis has been carried out. Once the analysis (or interaction) is complete, running buffer may enter the system to wash the sensor surface and prepare the system for a new assay.

It will be appreciated that the method of FIG. 18 can be implemented using more than two sample solutions, with each sample solution preferably separated from the preceding sample solution by a respective gas segment. The method of FIG. 18 may also be repeated as required, optionally with one or more characteristics modified in each successive repetition. For example, the order in which samples are injected and/or the environment in which interaction occurs (e.g. pH, concentration) can be altered as described above.

The above-described methodology opens up a wide array of potential applications and analyses, as will now be described in the following Implementations and Examples which provide a non-exhaustive list of applications to which the disclosed polyinjection and cycle subtraction methods and systems can be applied.

### Implementations

Below is provided a list of implementations in which the present inventors have identified the disclosed methods and systems may be particularly beneficial. It will be appreciated that this list is non-exhaustive and that the disclosed methods and systems may find utility in other implementations and contexts.
Revealing hidden binding signals and epitope binning: As described in detail above, the disclosed cycle subtraction methodology enables hidden binding signals to be revealed and unwanted “noise” from other association or dissociation signals to be removed. Epitope binning, where the competition between antibodies for antigen binding sites is analysed, is also facilitated. An implementation of this is described more fully below in Example 2.
Complex formation: the present methods and systems enable the build-up of complex formations at the sensor surface to be analysed more effectively. For example, analytes in first and second sample solutions may bind to one another to form a complex formation such as a complex of bound antibodies or a cascade reaction such as a blood clotting cascade. Such complexes may be unstable and be weakened or dissolve quickly in the presence of running buffer. Prior approaches which flow running buffer and prolong the time between samples (in the manner shown in FIG. 5) are thus unsuitable for performing these analyses effectively. The presently disclosed approaches enable the complexes to form and be analysed sufficiently quickly and without degrading the complex formation. This implementation is described more fully below in Example 1.
Revealing hidden binding signals and epitope binning: In addition, subsequent assays involving a subset of sample solutions may be conducted to reveal previously hidden binding characteristics. For example, a first assay involving a first set of sample solutions comprising three or more sample solutions may be conducted to obtain a first binding signal. A second assay involving a subset of the first set of sample solutions and a buffer sample may then be conducted to obtain a second binding signal. A difference binding signal may then be obtained by subtracting the second binding signal from the first binding signal. The difference binding signal can reveal previously hidden binding characteristics and interactions, and can also be used to identify whether analytes (e.g. antibodies) bind to the same or different epitopes (e.g. of an antigen) as one another. This implementation is described more fully below in Example 2.
Analysing ligand characteristics: the present methods and systems enable ligand behaviour in the presence of various analytes and environments at the sensor surface to be analysed more effectively. For example, binding to the ligand by analyte(s) in the presence of successive sample solutions having different pH values can be studied. This can also allow ligand conformation, refolding and stability characteristics to be studied more easily.
Preparation of different lipid environments: the present methods and systems enable study of lipid behaviour at the sensor surface to be analysed more effectively. For example, a first sample solution may comprise a membrane protein which is caught on the sensor surface (e.g. by a ligand). The second sample solution may comprise lipids configured to cover the membrane protein. This behaviour can be studied without any intervening running buffer which can interfere with the membrane protein.
Denaturation/renaturation experiments: the present methods and systems enable denaturation and renaturation of ligand proteins and nucleic acids (e.g. DNA and RNA) at the sensor surface to be analysed more effectively. In particular, interactions which involve fast switching between denaturing and renaturing conditions on the surface can be studied because there is no ‘down time’ during which running buffer flow is injected between samples.
Fast interactions: for similar reasons, the present methods and systems enable fast interactions at the sensor surface to be analysed more effectively. Examples include enzyme-substrate reactions which may take place in as little as 1 μs. In these contexts, the disclosed methods may provide an alternative to so-called stopped-flow experiments with quick introductions and removal of enzyme substrates, cofactors and products which are provided to influence the stability of binding of a molecule at the sensor surface. For similar reasons, analytes which naturally dissociate quickly can also be studied more effectively. Such fast-dissociating samples can also be locked onto a sensor surface in a procedure known as ‘dock and lock’, whereby the sensor surface is activated by reagents before introduction of the ligand and analytes. In this case, the time between said activation injection and ligand injection can be critical. Similar implementations involving cross-linking of the ligand with multi-step chemistry are also facilitated by the present methods and systems. Other examples of fast interactions include amine coupling, where activation of the sensory chip surface typically decays over time. The presently disclosed methods and systems allow faster assay runtimes resulting in increase of the immobilization level to mitigate this. Each of the above are facilitated by the disclosed methods and systems which enable fast interactions to be effectively analysed.
Antibody investigations: the present methods and systems enable the interaction of antibodies at the sensor surface to be analysed more effectively. In particular, several antibodies can be injected in sequence, and the behaviour of each investigated. This can shed light on how a given receptor interacts with different antibodies.
Lock-in experiments: by alternating injections of samples containing analytes and samples containing buffer, frequency analysis such as Fourier transformation can be employed to evaluate kinetics. This permits low signals (close to the measurement noise) to be detected and used in analysis. By changing the duration/volume of successive buffer samples (or buffer ‘pulses’), kinetics can also be assessed.
Analysis of sensitive analytes: some samples are not compatible with running buffer and so cannot be studied effectively using traditional methods where running buffer flows before and after each sample injection. Examples of such sensitive analytes include metal ions that precipitate buffer substances and cofactors. The present methods and systems enable such sensitive analytes to be studied at the sensor surface more effectively. For example, the sensitive substance may be protected by injection of other solutions before and after the sensitive solution. In this manner, the sensitive analyte is protected from running buffer flow and can be studied effectively.
Agonists and antagonists: the present methods and systems enable agonists and antagonists at the sensor surface to be analysed more effectively for example by successively injecting antagonists or agonists to map their behaviour.

It will be appreciated that one or more of the above implementations can be combined to provide yet further useful implementations and applications of the disclosed systems and methods.

### Examples

The below non-limiting examples set out two example implementations of the disclosed methods and systems in further detail.

### 1. Complex Formation

i. The ligand RBD (receptor binding domain, SARS-COV-2) in-house production was diluted from 2.53 mg/mL to 5 μg/mL with 10 mM sodium acetate pH 4.5.
ii. The ligand was amine coupled to Sensor Chip CM5 Cytiva by a pre-programed setting for a standard immobilization on Biacore system using the reagents in the Amine coupling kit from Cytiva. The ligand was injected for 7 min resulting in an immobilization level of 895 RU. Running buffer was HBS-EP+ from Cytiva. HBS-EP+ from Cytiva was used as running buffer and in buffer samples in all assays.
iii. Analyte dilution: A first analyte ACE2 (Angiotensin Converting Enzyme-2) stock, in-house production, was diluted from 1.1 mg/ml to 0.056 mg/mL with buffer to provide a first sample solution. A second analyte anti-RBD (clone CR3022) antibody from LifeSpan BioSciences was diluted from 1 mg/ml to 1.5 ug/mL with buffer to provide a second sample solution. A third analyte anti-Human IgG (Fc) from Cytiva was diluted from 0.5 mg/ml to 1.5 ug/mL with buffer to provide a third sample solution.
iv. A polyinject 3-segments command was used to inject the three sample solutions sequentially into the flow cell without any running buffer flow between the respective samples. A gas segment was provided between each sample solution in the flow cell to prevent mixing. Each gas segment was extracted shortly before reaching the sensor surface via another flow channel. The contact time for each sample injection was 120s at a flow rate of 30 μL/min. Injection of the three samples was followed by a dissociation time of 180s. The sensor chip was regenerated with 10 mM Glycine-HCl pH 1.5 from Cytiva after each with the regeneration solution having a contact time of 30 seconds.
v. A binding response signal was obtained from the assay, reflective of binding interactions at the sensor surface involving the first, second and third analytes. The binding response signal was suitable for complex formation analysis to determine how the samples interacted at the surface, in particular to determine whether binding of any analytes was dependent on the presence of any other analytes.
vi. The assay was repeated with a subset of the sample solutions from the first assay and a buffer sample solution. In this example, the sample solution containing anti-RBD (clone CR3022) was replaced with a buffer sample in the second assay. The resultant binding signal was subtracted from the initially obtained binding signal to give a difference signal, revealing the true binding characteristics of anti-RBD (clone CR3022) and identifying any associated dependencies or independence in binding. A third assay was then conducted, wherein the sample solution containing anti-Human IgG was replaced by a buffer sample. The resultant binding signal was once again subtracted from the initially obtained binding signal to give a difference signal, revealing the true binding characteristics of anti-Human IgG and identifying any associated dependencies or independence in binding. A fourth assay was then conducted, wherein the sample solution containing ACE2 (Angiotensin Converting Enzyme-2) was replaced by a buffer sample. The resultant binding signal was once again subtracted from the initially obtained binding signal to give a difference signal, revealing the true binding characteristics of ACE2 (Angiotensin Converting Enzyme-2) and identifying any associated dependencies or independence in binding.

### 2. Epitope Binning

i. Mouse Antibody Capture kit antibody from Cytiva was diluted from 1 mg/mL to 30 μg/mL with 10 mM sodium acetate pH 5.0 and amine coupled to Sensor chip CM5 by using the reagents in the Amine coupling kit from Cytiva. A pre-programed setting for standard and low immobilization on Biacore system was used for immobilization, i.e., a contact time of 7 min and 30 s respectively of EDC/NHS. A reference flow cell was left untreated. The immobilization procedure for the high ligand density, i.e. 7 min injection of EDC/NHS, resulted in 8657 RU and the immobilization procedure for low ligand density, i.e. 60s injection, resulted in 1007 RU. HBS-EP+ from Cytiva was used as running buffer and sample buffer in all assays.
ii. The antigen stock, Human Creatin Kinase (CKMB) from BioSpacific, was diluted from 1 mg/mL to 4.3 ug/mL with buffer to provide a first sample solution.
iii. Monoclonal mouse antibodies (mAb) against CKMB 1277, 1290 and 1285 in-house production were diluted to a final concertation of 100 nM with buffer to provide second, third and fourth sample solutions.
iv. A polyinject 3-segments command was used to inject samples of three of the four prepared solutions into the flow cell in various orders, and without any running buffer flow between the different samples as shown in the table below. A gas segment was provided between each sample solution in the flow cell to prevent mixing. Each gas segment was extracted shortly before reaching the sensor surface via another flow channel. Each antibody was tested against itself and against each other antibody. For each assay, a subsequent assay was performed with the antigen replaced by a buffer sample. This resulted in eighteen assays in total for each antibody pair as shown in Table 7 below. The contact time for each sample injection was 120s at a flow rate of 20 μL/min. In each assay, injection of the three samples was followed by a dissociation time of 30s. The sensor chip was regenerated with 10 mM Glycine-HCl PH 1.7 from Cytiva after each assay.

TABLE 7
Eighteen assays performed for each antibody
pair to investigate epitope competition.

| Assay | Sample A | Sample B | Sample C |
|---|---|---|---|
| 1 | 1277 | CKMB | 1277 |
| 2 | 1277 | Buffer sample | 1277 |
| 3 | 1277 | CKMB | 1285 |
| 4 | 1277 | Buffer sample | 1285 |
| 5 | 1277 | CKMB | 1290 |
| 6 | 1277 | Buffer sample | 1290 |
| 7 | 1285 | CKMB | 1285 |
| 8 | 1285 | Buffer sample | 1285 |
| 9 | 1285 | CKMB | 1277 |
| 10 | 1285 | Buffer sample | 1277 |
| 11 | 1285 | CKMB | 1290 |
| 12 | 1285 | Buffer sample | 1290 |
| 13 | 1290 | CKMB | 1290 |
| 14 | 1290 | Buffer sample | 1290 |
| 15 | 1290 | CKMB | 1285 |
| 16 | 1290 | Buffer sample | 1285 |
| 17 | 1290 | CKMB | 1277 |
| 18 | 1290 | Buffer sample | 1277 |

v. A binding response signal was obtained from each assay, reflective of binding interactions at the sensor surface involving the first, second and third samples in that respective assay. The binding response signals were subtracted from one another as appropriate and as described above in relation to FIG. 16a, to generate nine difference binding signals resembling those shown in FIG. 16a. These binding signals enabled determination of whether the respective antibodies competed for a same epitope of the antigen. It was established that antibodies 1290 and 1277 always compete, antibodies 1277 and 1285 as well as 1285 and 1290 do not compete.

### Variations

The above detailed description describes a variety of example arrangements for and methods for delivering sample solutions to a sensor surface of a biosensor. However, the described arrangements and methods are merely exemplary, and it will be appreciated by a person skilled in the art that various modifications can be made without departing from the scope of the appended claims. Some of these modifications will now be briefly described, however this list of modifications is not to be considered as exhaustive, and other modifications will be apparent to a person skilled in the art.

More generally, it should be appreciated that the number of steps and features shown in the figures is not intended to be limiting. Steps may be repeated as often as necessary and certain steps and features may be omitted.

In many of the above examples, a first assay is performed followed by a subsequent assay. The binding curve of the subsequent assay is then subtracted from the first assay. It will be appreciated that this ordering of assays is included merely for ease of understanding and can be altered. For example, a first assay can be performed, followed by a second assay. The binding signal of the first assay can then be subtracted from the binding signal of the second assay. That is, it is not a requirement that the second assay always be the one whose binding signal is subtracted to generate a difference signal. Rather, the only requirement is that one assay is performed with a set of sample solutions, and then another assay is performed using a subset of the sample solutions and a buffer sample. Then, the binding signal from the subset+buffer can be subtracted from the binding signal of the full set of samples. The order in which the assays is carried out is not important.

The above examples refer primarily to “injection” of samples into the flow channels of the biosensor system. In particular, in one preferred arrangement the first and second sample solutions are aspirated from respective first and second sample solution reservoirs and injected into the first channel for delivery to the sensor surface. However, it will be appreciated that this mechanism is non-limiting and samples can be introduced into the system in any suitable manner, such as by suction or other form of pressure which forces or sucks the sample solutions from their sample vials or reservoirs into the appropriate flow channel(s).

Similarly, movement or flow of sample solutions through flow channels of the system is typically controlled by one or more fluid flow pumps. In an example arrangement, each flow channel (for example the first and second flow channels discussed above) are operated under action of a respective pump. In that case, sample is sucked (or pushed) through the first channel by a first pump and then, at the appropriate moment, is sucked (or pushed) through the second channel by a second pump such that the gas segment can be removed. After removal of the gas segment, the first pump can once again take over to deliver the sample solutions to the sensor surface. While such an arrangement provides particularly precise control of the fluid flow, it will be appreciated that any suitable arrangement for causing sample solution to flow through the appropriate flow channels can be used, for example any suitable arrangement of pumps, valves, pistons and actuators.

The above examples describe how running buffer is provided from a running buffer reservoir using a running buffer pump. It will similarly be appreciated that any suitable arrangement of pumps, valves, pistons and actuators can be used to deliver the running buffer from the running buffer reservoir into the system.

As described above, the sensor surface may have a ligand immobilised thereto prior to analyte injection. In that case, binding of the analytes in the first and second (and potentially more) sample solutions to the ligand can be analysed. Alternatively, the first and second sample solutions can be injected without prior immobilisation of a ligand at the sensor surface. In that case, as noted above, the analyte in the first sample solution typically binds to the sensor surface and effectively acts as the ligand, capturing analyte from the second sample solution once it too reaches the surface. For ease of understanding, however, the term ‘ligand’ is herein reserved for a molecule which is introduced and immobilised to the sensor surface with the sole purpose of acting as an immobilized interactant rather than as an analyte whose binding response is measured. In some examples, the first sample can comprise a sensor preparation solution (e.g. an activation reagent) and the second solution can comprise a ligand to be attached to the activated sensor surface.

The above description uses the example of introducing a first sample followed by a second sample. It will be appreciated that in many cases it will be useful to repeat this process with one or more modified parameters. For example, the order in which the samples are introduced may be altered (e.g. sample A then B, followed by sample B then A). Alternatively/additionally, the environment in which the samples are introduced may be changed. For example, the pH value of one or more samples may be altered such that the effect of pH on the reaction can be analysed. Similarly, the concentration of at least one sample can be altered such that the effect of analyte concentration on the reaction can be analysed.

While the above description focusses on routines where each sample solution comprises an analyte of interest, one or more sample solutions may alternatively or additionally comprise a buffer solution, wash solution or sensor surface regeneration solution. By introducing these fluids as a sample solution, the operator has far greater control than when using, for example, continuous running buffer flow from the running buffer reservoir which typically administers unspecified or uncontrolled quantities of buffer solution. Providing these solutions as sample solutions allows discrete, highly controlled volumes of fluid to be provided to the sensor surface as part of an analysis to fulfil certain functions as part of the analysis, for example as shown in Example 2 above. As noted above, it is to be emphasised that a buffer sample is different to running buffer in that a buffer sample is provided from a sample reservoir (e.g. vial or microplate well) and is injected into the system in the same way as any other sample solution. It has a discrete, highly controlled volume and is injected for the purpose of analysing the behaviour of analytes at the sensor surface, for example as shown in Example 2 above. Running buffer, by contrast, is provided from a running buffer reservoir via a different mechanism (e.g. via a discrete running buffer pump) and has the purpose of washing out the analytes at the end of an assay and keeping the instrumentation wetted when no sample is flowing.

The volume of sample solution provided may be determined based on the desired (or target) duration during which time the sample is intended to be present at the sensor. This volume can be determined using the equation:

$$Volume = flow rate * time$$

where volume is the volume of sample solution introduced into the system, flow rate is the rate at which the sample solution passes over the sensor surface and time is the target duration during which time the sample solution is present at the sensor surface. For example, if the desired time at the sensor is 1 second and the flow rate is 100 μl per second, then it can be determined that the required volume of sample that needs to be injected is 100 μl.

The methods discussed above are introduced primarily in the context of SPR. However, it will be appreciated that any biosensor system having a sensor surface over which sample solutions can flow and where binding events can be detected is suitable for use with the disclosed methods. Such a biosensor may for example be based on evanescent wave sensing or another optical or non-optical mechanism for detecting binding or reaction events at the sensor surface.

When a gas segment is provided between samples and extracted, it is preferable to extract the gas segment sufficiently close to the second solution reaching the sensor surface that any mixing that occurs after removal of the gas segment is negligible. For example, in the implementation where first and second sample solutions are separated by a gas segment, the first sample solution may contact the sensor surface during a first time period, and the second sample solution may contact the sensor surface during a second time period. The first and second time periods may be referred to as the ‘contact times’ of the first and second samples. The delay between the end of the first time period and the start of the second time period (i.e. the gap between the two contact times) is preferably 40 seconds or less. This time threshold ensures that the disclosed methodology is suitable for analysing interactions involving analytes that dissociate or degrade quickly. For example, a first injected analyte in a first sample may bind to the sensor surface, but then dissociate soon after. Ensuring that the second sample arrives within 40 seconds or less ensures that for a wide range of implementations the second sample will arrive in time for the interaction of interest to occur at the sensor, before the first analyte dissociates. More preferably, the delay between the first and second sample is 30 seconds or less, 20 seconds or less, 10 seconds or less, or 1 second or less. These successively shorter time periods ensure that even very fast dissociating analytes can be analysed. The range of times is made possible through the methodologies and systems disclosed herein because there is no ‘down-time’ due to intermediate running buffer injection. The precise time between first and second samples arriving at the sensor can be determined by the length of flow channel that the samples flow through between gas segment extraction and the sensor, for example. The disclosed methodologies and available range of times between samples means the assay environment can be developed around and modified to suit particular analytes of interest.

### Computer System

Turning finally to FIG. 19, FIG. 19 shows a schematic and simplified representation of a computer apparatus 1801 which can be used to perform the methods described herein, either alone, in combination with other computer apparatuses or as part of a ‘cloud’ computing arrangement. For example, computer apparatus 1801 may form part of, or be connected (wirelessly or via a wired connection) to, analysis system 300 and may be configured to cause analysis system 300 to perform the various methods disclosed herein. Computer apparatus 1801 may also perform analysis on the readings or information produced by the sensor of analysis system 300.

The computer apparatus 1801 in the example shown comprises various data processing resources such as a processor 1802 (in particular, a hardware processor) coupled to a central bus structure. Also connected to the bus structure are further data processing resources such as memory 1804. A display adapter 1806 connects a display device 1808 to the bus structure. One or more user-input device adapters 1810 connect a user-input device 1812, such as a keyboard and/or a mouse to the bus structure. One or more communications adapters 1814 are also connected to the bus structure to provide connections to other computer systems 1801 and other networks. Computer apparatus 1801 may be a local computer or a server. It may be a standalone element or may be part of existing computer hardware for use in a sample analysis laboratory.

In operation, the processor 1802 of computer system 1801 executes a computer program comprising computer-executable instructions that may be stored in memory 1804. When executed, the computer-executable instructions may cause the computer system 1801 to cause a sample analysis system 300 to perform one or more of the methods described herein. The results of the processing (for example a sensorgram as shown in FIG. 2) may be displayed to a user via the display adapter 1806 and display device 1808. User inputs for controlling the operation of the computer system 1801 may be received via the user-input device adapters 1810 from the user-input devices 1812.

It will be apparent that some features of computer system 1801 shown in FIG. 19 may be absent in certain cases. For example, one or more of the plurality of computer apparatuses 1801 may have no need for display adapter 1806 or display device 1808. This may be the case, for example, for particular computer apparatuses 1801 which are used only for their processing capabilities and do not need to display information directly to users. Similarly, user input device adapter 1810 and user input device 1812 may not be required. In its simplest form, computer apparatus 1801 comprises processor 1802 and memory 1804.

As noted above, the described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g., for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

While various specific combinations of components and method steps have been described, these are merely examples. Components and method steps may be combined in any suitable arrangement or combination. Components and method steps may also be omitted to leave any suitable combination of components or method steps.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations and examples are to be considered in all respects only as illustrative and not restrictive. It will be appreciated that variations of the described implementations may be made without departing from the scope of the disclosure. It will also be apparent that there are many variations that have not been described, but that fall within the scope of the appended claims.

### CLAUSES

Also disclosed herein are the following clauses:
1. A method of delivering a plurality of sample solutions to a sensor surface for analysis, comprising:
   a) introducing a first sample solution into a first channel, the first channel configured to deliver sample solution to the sensor surface;
   b) introducing a second sample solution into the first channel, wherein the second sample solution is separated from the first sample solution in the first channel by a gas segment;
   c) flowing the first sample solution, second sample solution and gas segment through the first channel towards the sensor surface;
   d) prior to the gas segment reaching the sensor surface, extracting the gas segment from the first channel via a second channel such that the gas segment does not contact the sensor surface;
   e) flowing the first sample solution and the second sample solution over the sensor surface; and
   f) detecting the presence or absence of binding of analyte from at least one of the sample solutions at the sensor surface.
2. The method of clause 1, wherein no running buffer solution is passed over the sensor surface throughout steps a)-f).
3. The method of any preceding clause, wherein introducing the first and second sample solutions into the first channel comprises aspirating the first and second sample solutions from respective first and second sample solution reservoirs and injecting the sample solutions into the first channel.
4. The method of any preceding clause, wherein a first pump is configured to introduce sample solutions into the first channel and cause the sample solutions to flow through the first channel and over the sensor surface.
5. The method of any preceding clause, wherein, prior to step a) and/or after step f), running buffer obtained from a running buffer reservoir is caused to pass over the sensor surface by a second pump.
6. The method of any preceding clause, wherein the gas segment is extracted at most 40 seconds prior to the second sample solution reaching the sensor surface.
7. The method of any preceding clause, wherein the method is performed for three or more sample solutions, optionally wherein each sample solution is separated from any preceding sample solution in the first channel by a respective gas segment.
8. The method of any preceding clause, wherein the sensor surface has a ligand immobilized thereto and step f) comprises detecting the presence or absence of binding of analyte from at least one sample solution to the ligand on the sensor surface.
9. The method of any preceding clause, wherein step f) comprises identifying whether an analyte comprised in one of the sample solutions binds to an analyte comprised in another of the sample solutions that has previously bound to the sensor surface.
10. The method of any preceding clause, wherein the first and second sample solutions are introduced into the first channel in a first order, and wherein the method further comprises:

repeating the method steps with the first and second sample solutions introduced into the first channel in a second order different to the first order.
11. The method of any preceding clause, wherein the first sample solution comprises a first analyte and has a first pH value, and wherein the second sample solution has a second pH value and does not comprise an analyte.
12. The method of any preceding clause, wherein the first and second sample solutions have first and second respective concentrations, and wherein the method further comprises:

modifying the concentration of at least one of the first and second sample solutions and repeating the method steps with the modified solution(s).
13. The method of any preceding clause, wherein at least one sample solution comprises: buffer; an antibody; an antigen; an enzyme; an agonist; an antagonist; a wash solution; or a sensor surface regeneration solution.
14. The method of any preceding clause, wherein the first sample solution contacts the sensor surface during a first time period, the second sample solution contacts the sensor surface during a second time period, and the delay between the end of the first time period and the start of the second time period is 40 seconds or less.
15. The method of any preceding clause, wherein the first sample solution comprises a membrane protein and the second sample solution comprises a lipid configured to at least partially cover the membrane protein.
16. The method of any preceding clause, further comprising identifying a complex that is built up on the sensor surface, said complex comprising analytes from at least two of the sample solutions.
17. The method of any preceding clause, further comprising identifying whether analyte comprised in at least one of the sample solution binds to a same or different epitope as analyte comprised in another of the sample solutions.
18. The method of any preceding clause, wherein step f) is based on evanescent wave sensing.
19. The method of any preceding clause, wherein step f) is based on surface plasmon resonance (SPR).
20. The method of any preceding clause, wherein the gas segment comprises air.
21. The method of any preceding clause, wherein the first and second channels form a substantially T-shaped junction.
22. A sample analysis system comprising:
   a first channel;
   a second channel; and
   a sensor surface, wherein the sample analysis system is configured to perform the steps of:
   a) introducing a first sample solution into the first channel, the first channel configured to deliver sample solution to the sensor surface;
   b) introducing a second sample solution into the first channel, wherein the second sample solution is separated from the first sample solution in the first channel by a gas segment;
   c) flowing the first sample solution, second sample solution and gas segment through the first channel towards the sensor surface;
   d) prior to the gas segment reaching the sensor surface, extracting the gas segment from the first channel via the second channel such that the gas segment does not contact the sensor surface;
   e) flowing the first sample solution and the second sample solution over the sensor surface; and
   f) detecting the presence or absence of binding of analyte from at least one of the sample solutions at the sensor surface.
23. A computer configured to cause a sample analysis system to perform the method of any of clauses 1-21.
24. A computer-readable storage medium comprising instructions which, when executed by a computer, cause the computer to cause a sample analysis system to perform the method of any of clauses 1-21.

1. A method of delivering a plurality of sample solutions to a sensor surface (102) for analysis, comprising:
a) introducing (902) a first sample solution (502) into a first channel (506), the first channel configured to deliver sample solution to the sensor surface (102);
b) introducing (904) a second sample solution (504) into the first channel;
c) flowing (906) the first sample solution and the second sample solution (502, 504) through the first channel (506) and over the sensor surface (102); and
d) detecting (912) the presence or absence of binding of analyte from at least one of the sample solutions (502, 504) at the sensor surface,
wherein no running buffer solution is passed over the sensor surface (102) throughout steps a)-d).

2. The method of claim 1, wherein the second sample solution (502) is separated from the first sample solution (504) in the first channel (506) by a gas segment (503).

3. The method of claim 2, further comprising, prior to the gas segment (503) reaching the sensor surface, extracting (908) the gas segment from the first channel (506) via a second channel (508) such that the gas segment (503) does not contact the sensor surface (102).

4. The method of any preceding claim, wherein introducing the first and second sample solutions (502, 504) into the first channel (506) comprises aspirating the first and second sample solutions from respective first and second sample solution reservoirs and injecting the sample solutions into the first channel.

5. The method of any preceding claim, wherein a first pump (404) is configured to introduce sample solutions into the first channel and cause the sample solutions (502, 504) to flow through the first channel (506) and over the sensor surface (102).

6. The method of any preceding claim, wherein, prior to step a) and/or after step d), running buffer obtained from a running buffer reservoir is caused to pass over the sensor surface (102) by a second pump.

7. The method of any preceding claim, wherein the gas segment (103) is extracted at most 40 seconds prior to the second sample solution (504) reaching the sensor surface (102).

8. The method of any preceding claim, wherein the method is performed for three or more sample solutions, optionally wherein each sample solution is separated from any preceding sample solution in the first channel (506) by a respective gas segment (103).

9. The method of any preceding claim, wherein the sensor surface (102) has a ligand immobilized thereto and step d) comprises detecting the presence or absence of binding of analyte from at least one sample solution (502, 504) to the ligand on the sensor surface (102).

10. The method of any preceding claim, wherein step d) comprises identifying whether an analyte comprised in one of the sample solutions (502, 504) binds to an analyte comprised in another of the sample solutions that has previously bound to the sensor surface (102).

11. The method of any preceding claim, wherein the first and second sample solutions (502, 504) are introduced into the first channel in a first order, and wherein the method further comprises:
repeating the method steps with the first and second sample solutions (502, 504) introduced into the first channel in a second order different to the first order.

12. The method of any preceding claim, wherein the first sample solution (502) comprises a first analyte and has a first pH value, and wherein the second sample (504) solution has a second pH value and does not comprise an analyte.

13. The method of any preceding claim, wherein the first and second sample solutions (502, 504) have first and second respective concentrations, and wherein the method further comprises:
modifying the concentration of at least one of the first and second sample (502, 504) solutions and repeating the method steps with the modified solution(s).

14. The method of any preceding claim, wherein at least one sample solution (502, 504) comprises: buffer; an antibody; an antigen; an enzyme; an agonist; an antagonist; a wash solution; or a sensor surface regeneration solution.

15. The method of any preceding claim, wherein the first sample solution (502) contacts the sensor surface (102) during a first time period, the second sample solution (504) contacts the sensor surface (102) during a second time period, and the delay between the end of the first time period and the start of the second time period is 40 seconds or less.

16. The method of any preceding claim, wherein the first sample solution (502) comprises a membrane protein and the second sample solution comprises a lipid configured to at least partially cover the membrane protein.

17. The method of any preceding claim, further comprising identifying a complex that is built up on the sensor surface (102), said complex comprising analytes from at least two of the sample solutions (502, 504).

18. The method of any preceding claim, further comprising identifying whether analyte comprised in at least one sample solution (502, 504) binds to a same or different epitope as analyte comprised in another of the sample solutions.

19. The method of any preceding claim, wherein step d) is based on evanescent wave sensing.

20. The method of any preceding claim, wherein step d) is based on surface plasmon resonance (SPR).

21. The method of any preceding claim, wherein the gas segment (103) comprises air.

22. The method of any preceding claim, wherein the first and second channels (506, 508) form a substantially T-shaped junction.

23. A sample analysis system comprising:
a first channel (506); and
a sensor surface (102),
wherein the sample analysis system is configured to perform the steps of:
a) introducing a first sample solution (502) into the first channel (506), the first channel (506) configured to deliver sample solution to the sensor surface (102);
b) introducing a second sample solution (504) into the first channel (506);
c) flowing the first sample solution (502) and the second sample solution (504) through the first channel (506) and over the sensor surface (102); and
d) detecting the presence or absence of binding of analyte from at least one of the sample solutions (502, 504) at the sensor surface (102),
wherein no running buffer solution is passed over the sensor surface throughout steps a)-d).

24. A method of assaying multiple sets of sample solution, comprising:
providing (1402) a first set of sample solutions, wherein the first set of sample solutions comprises at least three sample solutions;
obtaining a first binding signal (1406) by performing an assay (1404) with the first set of sample solutions during which each sample solution in the first set of sample solutions is flowed sequentially over a sensor surface;
providing a second set of sample solutions (1408), wherein the second set of sample solutions comprises a subset of the first set of sample solutions and a buffer sample solution;
obtaining a second binding signal (1412) by performing an assay (1410) with the second set of sample solutions during which each sample solution in the second set of sample solutions is flowed sequentially over the sensor surface; and
generating a third binding signal (1416) by subtracting (1414) the second binding signal from the first binding signal.

25. The method of claim 24, wherein providing the first and/or second set of sample solutions comprises introducing the respective sample solutions into a channel configured to deliver sample solution to the sensor surface.

26. The method of claim 25, wherein each sample solution introduced into the channel is separated from any preceding sample solution in the channel by a respective gas segment.

27. The method of claim 26, further comprising, prior to each gas segment reaching the sensor surface, extracting the respective gas segment from the channel via another channel such that each respective gas segment does not contact the sensor surface.

28. The method of any of claims 25-27, wherein introducing the first and/or second set of sample solutions comprises aspirating the respective sample solutions from respective sample solution reservoirs and injecting the sample solutions into the channel.

29. The method of any of claims 24 to 28, wherein obtaining the first and/or second binding signal comprises detecting the presence or absence of binding involving analytes from the respective sample solutions at the sensor surface.

30. The method of claim 29, wherein the sensor surface has a ligand immobilized thereto and obtaining the first and/or second binding signal comprises detecting the presence or absence of binding of analyte from at least one sample solution to the ligand on the sensor surface.

31. The method of claim 29 or 30, wherein obtaining the first and/or second binding signal comprises identifying whether an analyte comprised in one of the sample solutions binds to an analyte comprised in another of the sample solutions that has previously bound to the sensor surface.

32. The method of any of claims 24 to 31, wherein the first set of sample solutions comprises a first sample solution and a second sample solution, wherein when the second sample solution reaches the sensor surface the first sample solution generates a dissociation signal equal or greater than an association signal generated by the second sample solution, and wherein the second set of sample solutions does not comprise the second sample solution.

33. The method of any of claims 24 to 32, wherein the first set of sample solutions comprises a first sample solution and a second sample solution, wherein analyte in the second sample solution is capable of binding to analyte in the first sample solution and to a ligand on the sensor surface, and wherein the second set of sample solutions does not comprise the first sample solution.

34. The method of any of claims 24 to 33, wherein the first set of sample solutions comprises a first antibody, an antigen, and a second antibody.

35. The method of claim 34, wherein the second set of sample solutions does not comprise the antigen.

36. The method of claim 34 or 35, wherein the second set of sample solutions does not comprise the second antibody.

37. The method of any of claims 24 to 36 further comprising determining, based on the third binding signal, whether binding of analyte in a second sample solution in the first set of sample solutions is dependent on the presence of analyte from a first sample solution in the first set of sample solutions.

38. The method of any of claims 24 to 37 further comprising determining, based on the third binding signal, whether analyte in a first sample solution in the first set of sample solutions binds to a same epitope as analyte in a second sample solution in the first set of sample solutions.

39. The method of any of claims 24 to 38, wherein at least one sample solution comprises: buffer; an antibody; an antigen; an enzyme; an agonist; an antagonist; a wash solution; or a sensor surface regeneration solution.

40. The method of any of claims 24 to 39, wherein performing an assay with the first and/or second set of sample solutions is based on evanescent wave sensing.

41. The method of any of claims 24 to 40, wherein performing an assay with the first and/or second set of sample solutions is based on surface plasmon resonance (SPR).

42. A sample analysis system, comprising:
a sensor (102) having a surface over which sample solution (502, 504) can flow; and
at least one channel (506, 508) configured to deliver sample solution to the sensor surface;
wherein the sample analysis system is configured to:
provide a first set of sample solutions to the at least one channel, wherein the first set of sample solutions comprises at least three sample solutions;
perform an assay wherein each sample solution in the first set of sample solutions is flowed sequentially over the sensor surface (102), in order to obtain a first binding signal;
provide a second set of sample solutions to the at least one channel, wherein the second set of sample solutions comprises a subset of the first set of sample solutions and a buffer sample solution;
perform an assay wherein each sample solution in the second set of sample solutions is flowed sequentially over the sensor surface (102), in order to obtain a second binding signal; and
generate a third binding signal by subtracting the second binding signal from the first binding signal.

43. A computer (600) configured to cause a sample analysis system to perform the method of any of claims 1-22 or 24 to 41.

44. A computer-readable storage medium comprising instructions which, when executed by a computer, cause the computer to cause a sample analysis system to perform the method of any of claims 1-22 or 24 to 41.
